(19) United States
(12) Patent Application Publication
(10) Pub. No.: US 2022/0338434 A1
(43) Pub. Date: Oct. 27, 2022

(54) GENERATION OF HAPLOIDS BASED ON MUTATION OF SAD2

(71) Applicant: Leibniz-Institut für Pflanzengenetik und Kulturpflanzenforschung (IPK), Seeland/OT Gatersleben (DE)

(72) Inventors: Dmitri Demidov, Seeland/OT Gatersleben (DE); Andreas Houben, Seeland/OT Gatersleben (DE); Jörg Fuchs, Seeland/OT Gatersleben (DE); Oda Weiss, Seeland/OT Gatersleben (DE); Karla Meier, Seeland/OT Gatersleben (DE); Inna Lermontova, Seeland/OT Gatersleben (DE)

(21) Appl. No.: 17/762,724

(22) PCT Filed: Sep. 22, 2020
(86) PCT No.: PCT/EP2020/076439

§ 371 Date: Mar. 22, 2022

(30) Foreign Application Priority Data
Sep. 23, 2019 (EP) 19198975.5

(51) Int. Cl. A01H 1/08; A01H 5/10; C12N 15/82

(52) U.S. Cl. CPC A01H 1/08; A01H 5/10; C12N 15/8216

(57) ABSTRACT

The invention identifies plants carrying certain mutations, which can be used as haploid inducers or doubled haploid inducers in plant breeding. According to the invention, significant haploid induction and even doubled haploid induction rates can be observed when at least one functional mutation within a SAD-homology domain is present, wherein the functional mutation affects the expression of certain SAD-homology consensus sequences. The present invention further provides haploid and doubled haploid plants, which are obtained by contacting a first gamete from a plant as identified according to the invention with a second gamete from a plant expressing a wild-type SAD-homology domain to generate a zygote. Furthermore, the present invention relates to a method for identifying a plant having the activity of a haploid inducer or a doubled haploid inducer in a plant population.

### CROSS-REFERENCE TO RELATED APPLICATIONS

This application is a U.S. National Stage Application under 35 U.S.C. 371 of International Application No. PCT/EP2020/076439, filed Sep. 22, 2020, which claims priority to European Patent Application No. 19198975.5, filed Sep. 23, 2019. The contents of each of which are hereby incorporated by reference in their entirety into the present disclosure.

### SEQUENCE LISTING

The instant application contains a sequence listing which has been submitted in ascii format via EFS-Web and is hereby incorporated by reference in its entirety. Said ascii copy, created Mar. 22, 2022, is named 208712_sequence_listing and is 193,539 bytes in size.

### TECHNICAL FIELD

The invention identifies plants carrying certain mutations, which can be used as haploid inducers or doubled haploid inducers in plant breeding. According to the invention, significant haploid induction and even doubled haploid induction rates can be observed when at least one functional mutation within a SAD-homology domain is present in the plant, wherein the functional mutation affects the expression of certain SAD-homology consensus sequences. The present invention further provides haploid and doubled haploid plants, which are obtained by contacting a first gamete from a plant as identified according to the invention with a second gamete from a plant expressing a wild-type SAD-homology domain to generate a zygote. Corresponding methods for generating haploid plant cells or for directly generating doubled haploid plant cells without the need for a chromosome doubling step are also provided. Furthermore, the present invention relates to a method for identifying a plant having the activity of a haploid inducer or a doubled haploid inducer in a plant population.

### BACKGROUND

Extensive population screening, even with the latest molecular breeding tools, is both laborious and costly. Efficient plant breeding aims at selecting favourable traits in a germplasm of interest. Heterogeneous breeding material in usually complex plant genomes significantly hampers the plant breeding process so that the selection process may be very cumbersome and time and cost intensive.

The generation and use of haploids is one of the most powerful biotechnological means to improve cultivated plants. The advantage of haploids for breeders is that homozygosity can be achieved already in the first generation after dihaploidization or more generally doubled haploidization by chemical means or by spontaneous chromosome doubling, creating doubled haploid plants, without the need of laborious backcrossing steps to obtain a high degree of homozygosity. Furthermore, the value of haploids in plant research and breeding lies in the fact that the founder cells of doubled haploids are products of meiosis, so that resultant populations constitute pools of diverse recombinant and at the same time genetically fixed individuals. The generation of doubled haploids thus provides not only perfectly useful genetic variability to select from with regard to crop improvement, but is also a valuable means to produce mapping populations, recombinant inbreds as well as instantly homozygous mutants and transgenic lines.

Haploid plants can be obtained by interspecific crosses, in which one parental genome is eliminated after fertilization. It was shown that genome elimination after fertilization could be induced by modifying a centromere protein, the centromere-specific histone CENH3 in Arabidopsis thaliana (Ravi and Chan, Haploid plants produced by centromere-mediated genome elimination, Nature, Vol. 464, 2010, 615-619). With the modified haploid inducer lines, haploidization occurred in the progeny when a haploid inducer plant was crossed with a wild-type plant. Interestingly, the haploid inducer line was stable upon selfing, suggesting that a competition between modified and wild-type centromere in the developing hybrid embryo results in centromere inactivation of the inducer parent and consequently in uniparental chromosome elimination.

Haploidization is thus an efficient tool to reduce the length of breeding schemes to create new cultivars of interest. Phenomena like gynogenesis and androgenesis induction can be important during haploid induction. Whilst gynogenesis is a process in which the embryo genome originates exclusively from female origin, following embryogenesis stimulation by a male gamete, androgenesis refers to the development of embryos that contain only the male nuclear genetic background.

In diverse publications, e.g. Portemer et al. 2015 (Portemer, V., Renne, C., Guillebaux, A., & Mercier, R. (2015). Large genetic screens for gynogenesis and androgenesis haploid inducers in Arabidopsis thaliana failed to identify mutants. Frontiers in plant science, 6, 147.), large genetic screens have thus been conducted in an attempt to screen for gynogenesis or androgenesis haploid inducer events. Still, these screens failed to identify specific mutants or a direct correlation between a mutated gene and its haploid induction potential.

To define a more systematic approach, the inventors of the present invention tested selected T-DNA mutants of Arabidopsis thaliana for their ability to induce the generation of haploids in crosses with wild-type. This screening has resulted in the selection of a few candidate genes, whereby the most promising candidates are SAD2 (AT2G31660) and SAD1 gene (AT3G59020). Based on these findings, large in silico screens were performed to identify consensus sequences based on the SAD family of proteins in major agricultural crop plants to provide proof-of-principle of the broad applicability of SAD mutants in (di)haploid induction, which findings were in turn confirmed by inducer screens with selected plant material. In view of the fact that the nomenclature of the sad genes derived from plants strongly varies and is driven by the specific phenotype the relevant authors initially attributed to the characterized mutants, all genes or proteins having a certain sequence identity to SAD2 according to SEQ ID NO: 1 (nucleic acid sequence) or SEQ ID NO: 2 (amino acid sequence) will be referred to as SAD-homology (nucleic acid or amino acid) sequence or protein herein, respectively.

SAD2 (Sensitive to ABA (abscisic acid) and Drought2) was described in Verslues et al. (2006). Mutation of SAD2, an importin β-domain protein in Arabidopsis, alters abscisic acid sensitivity. The Plant Journal, 47(5), 776-787.), where the authors studied sad2-1 mutant Arabidopsis plant. SAD2 was found to encode an importin beta-domain family protein likely to be involved in nuclear transport. SAD2 was expressed at a low level in all tissues examined except flowers, but SAD2 expression was not inducible by ABA or stress. Subcellular localization of GFP-tagged SAD2 showed a predominantly nuclear localization, consistent with a role for SAD2 in nuclear transport. Knockout of the closest importin beta homolog of SAD2 in Arabidopsis (sad2-1 mutant) indicates that SAD2 plays a specific role in ABA signaling. Analysis of ABA and stress sensitivity in double mutants of sad2-1 and sad1 suggested that SAD2 acts upstream of or has additive effects with the sad1 gene. Gao et al. ((2008). SAD2 in Arabidopsis functions in trichome initiation through mediating GL3 function and regulating GL1, TTG1 and GL2 expression. Journal of integrative plant biology, 50(7), 906-917.) report that the importin beta-like protein SAD2, is required for trichome initiation. Mutations in SAD2 disrupted trichome initiation resulting in reduced trichome number, but had no effect on trichome development or root hair number and development. Thereby it seems that SAD2 is in the same pathway as two transcription factors (GLABROUS1 (GL1) and GLABRA3 (GL3)).

SAD1 and SAD2, by alignments, have been shown to comprise a so-called Cse1 domain. From previous studies on SAD1 and SAD2 in other contexts, the Cse1 domain is thought to be crucial for the functionality of SAD1 and SAD2 being involved in the exchange of macromolecules between the nucleus and cytoplasm which takes place through nuclear pore complexes within the nuclear membrane. Active transport of large molecules through these pore complexes require carrier proteins, called karyopherins (importins and exportins), which shuttle between the two compartments. Still, the Cse1 family of proteins is found in a variety of different taxa from yeasts to mammals and in quite different classes of proteins. Therefore, it was an object to more precisely define those regions within SAD1 and SAD2 which could be attributed to the effects observed in this invention to identify reliable consensus sequences responsible for the specific new functional effects of SAD1 and SAD2 described herein.

So far, the SAD family of proteins in plants has not yet been associated with haploid or even spontaneous doubled haploid or dihaploid induction events at all. As detailed above, doubled haploid induction naturally usually occurs only in very rare events. Therefore, any induced haploid system requires an additional step of chromosome doubling, e.g. by chemical means. Many of those chemicals traditionally used in chromosome doubling, e.g. colchicine, are rather toxic. Therefore, there exists a great need in defining strategies to obtain doubled haploids from induced haploids in a time-efficient manner preferably without the need of additional chromosome doubling steps further complicating rapid breeding.

Therefore, it was an object of the present invention to explore the haploid induction potential of new mutant plant genes and the corresponding proteins. In particular, it was an object to define promising mutants or knock-outs based on the systematic manipulation of new candidate genes/proteins suitable to be used as maternal and/or paternal haploid inducers for providing effective new means for haploid, or even doubled haploid or dihaploid, induction in relevant agricultural crop plants to expedite breeding programs.

### SUMMARY OF THE INVENTION

In a first aspect, the present invention relates to the use of a plant seed, plant cell or part of a plant comprising a nucleotide sequence encoding an amino acid sequence comprising a SAD-homology domain, wherein the SAD-homology domain comprises at least one functional mutation, wherein the at least one functional mutation results in a decreased or abolished expression of the amino acid sequence comprising a SAD-homology domain in comparison to the cognate wild-type amino acid sequence, wherein the functional mutation affects the expression of a SAD-homology domain consensus sequence as set forth in any one of SEQ ID NOs: 19, 25 and 26, or an amino acid sequence having at least 90%, 91%, 92%, 93%, 94%, 95%, 96%, 97%, 98%, or 99% sequence identity to the sequence set forth in any one of SEQ ID NOs: 19, 25 and 26 for the induction of a haploid or a doubled haploid plant.

In one embodiment of the various aspects of the present invention, the nucleotide or amino acid sequence comprising a SAD-homology domain is selected from a sequence as set forth in any one of SEQ ID NOs: 1 to 4, 6 to 18 and 20 to 24, or a nucleotide or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in any one of SEQ ID NOs: 1 to 4, 6 to 18 and 20 to 24.

In another embodiment of the various aspects of the present invention, the functional mutation is selected from a point mutation in the coding sequence, or in a regulatory sequence, a frameshift mutation, an insertion mutation, or a knock-out or knock-down of/in a nucleic acid sequence encoding the sequence comprising a SAD-homology domain or a regulatory sequence thereof, wherein the functional mutation results in a loss of expression or loss of transcription.

In one embodiment of the various aspects of the present invention, generating a zygote from the plant, seed, or plant cell and a wild-type plant or a plant expressing a wild-type SAD-homology domain comprising amino acid sequence yields at least 0.5%, preferably at least 1.0%, at least 2.0%; at least 3.0%, at least 4.0% at least 5.0%, at least 6.0%, at least 7.0% at least 8.0%, at least 9.0%, at least 10.0%, at least 11.0%, at least 12.0%, at least 13.0%, at least 14.0% or at least 15.0% haploid progeny.

In a further embodiment according to the various aspects of the present invention, generating a zygote from the plant, seed, or plant cell and a wild-type plant or a plant expressing a wild-type SAD-homology domain comprising amino acid sequence yields at least 0.5%, preferably at least 1.0%, at least 2.0%; at least 3.0%, at least 4.0% at least 5.0%, at least 6.0%, at least 7.0%, at least 8.0%, at least 9.0%, at least 10.0%, at least 11.0%, at least 12.0%, at least 13.0%, at least 14.0% or at least 15.0% doubled haploid progeny.

In another embodiment of the various aspects of the present invention, the plant, seed, or plant cell is a paternal and/or maternal haploid or doubled haploid inducer.

In yet another embodiment of the various aspects of the present invention, the nucleotide and/or amino acid sequence encoding or comprising the SAD-homology domain comprises at least one endogenous gene or at least one transgene.

According to another aspect, the present invention relates to a haploid plant obtained by contacting a first gamete from a plant as defined in any of the embodiments above with a second a gamete from a plant expressing an amino acid sequence comprising a wild-type SAD-homology domain to generate a zygote.

According to a further aspect, the present invention relates to a doubled haploid plant obtained by converting the haploid plant according to the aspect described above into a doubled haploid plant, preferably a spontaneous doubled haploid plant directly obtained by contacting a first gamete from a plant as defined in any of the embodiments above with a second a gamete from a plant expressing an amino acid sequence comprising a wild-type SAD-homology domain.

According to one aspect, the present invention relates to a nucleic acid sequence encoding a consensus sequence as set forth in any one of SEQ ID NOs: 19, 25 and 26, or a sequence having at least 90%, 91%, 92%, 93%, 94%, 95%, 96%, 97%, 98%, or 99% sequence identity thereto, or a nucleotide sequence or an amino acid sequence as set forth in any one of SEQ ID NOs: 1 to 4, 6 to 18 and 20 to 24, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

According to another aspect, the present invention relates to a method of generating a haploid or doubled haploid plant cell, comprising the steps of:
a) providing a first gamete from a plant having the activity of a haploid or doubled haploid inducer as defined in any of the embodiments described above;
b) contacting the first gamete from step a), preferably a male gamete, with a second gamete of a wild-type plant, or of a plant comprising a wild-type SAD-homology domain comprising amino acid sequence or a sequence encoding the same to generate a F1 zygote;
c1) obtaining a haploid cell by elimination of the chromosomes of the plant having the activity of a haploid inducer from the F1 zygote, or
c2) directly obtaining a doubled haploid F1 zygote cell.

In one embodiment the method described above comprises, in addition to steps a) to c) the following steps:
d1) growing the haploid cell under conditions to obtain a haploid plant or a part thereof; and
e1) obtaining a haploid plant or part thereof; or
d2) growing the doubled haploid cell under conditions to obtain a doubled haploid plant or a part thereof; and
e2) obtaining a doubled haploid plant or part thereof.

In one aspect, the present invention relates to a method of directly generating a doubled haploid plant cell comprising the steps of:
a) providing a first gamete from a plant having the activity of a doubled haploid inducer as defined in any of the embodiments described above,
b) contacting the first gamete from step a), preferably a male gamete, with a second gamete of a wild-type plant, or of a plant comprising a wild-type SAD-homology domain comprising amino acid sequence or a sequence encoding the same to generate a F1 zygote;
c) directly obtaining a doubled haploid cell as F1 zygote.

In one embodiment, the method of directly generating a doubled haploid plant cell described above, further comprises, in addition to steps a) to c), the following steps:
d) growing the doubled haploid cell; and
e) obtaining a doubled haploid plant, part thereof, or seed.

According to another aspect, the present invention relates to a method for identifying a plant having activity of a haploid inducer or a doubled haploid inducer comprising the step of:
a) providing a population of plants comprising a nucleotide sequence encoding an amino acid sequence comprising a SAD-homology domain;
b) screening the plant population for the presence of at least one functional mutation in the SAD-homology domain, wherein the at least one functional mutation results in a decreased or abolished expression of the amino acid sequence comprising a SAD-homology domain in comparison to the cognate wild-type amino acid sequence, wherein the functional mutation affects the expression of a SAD-homology domain consensus sequence as set forth in any one of SEQ ID NOs: 19, 25 and 26, or an amino acid sequence having at least 90%, 91%, 92%, 93%, 94%, 95%, 96%, 97%, 98%, or 99% sequence identity to the sequence set forth in any one of SEQ ID NOs: 19, 25 and 26; and
c) optionally, obtaining a plant having activity of a haploid or a doubled haploid inducer.

The present invention will now be disclosed in more detail based on the disclosure below, the non-limiting Examples as well as the attached Sequence Listing.

### Definitions

The terms “plant” or “plant cell” or “part of a plant” as used herein refer to a plant organism, a plant organ, differentiated and undifferentiated plant tissues and derivatives and progeny thereof. Plant cells include without limitation, for example, cells from seeds, from mature and immature cells or organs, including embryos, meristematic tissues, seedlings, callus tissues in different differentiation states, leaves, flowers, roots, shoots, male or female gametophytes, sporophytes, pollen, pollen tubes and microspores, protoplasts, macroalgae and microalgae. A part of a plant includes leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cut-tings.

A “SAD-homology domain” in the context of the present disclosure refers to amino acid sequence which comprises at least one of the consensus sequences as set forth in any one of SEQ ID NOs: 19, 25 and 26 or which comprises at least one amino acid sequence having at least 90% sequence identity to any one of the sequences set forth in SEQ ID NOs: 19, 25 and 26. In different plants, SAD-homology domains may have different names and varying degrees of homology among each other, i.e. a range from 70 to 90% sequence identity is found. Examples for certain plants are given in SEQ ID NOs: 1 to 4, 6 to 8 and 20 to 24. In the context of the present disclosure, a SAD-homology domain comprises at least one of the consensus sequences of SEQ ID NOs: 19, 25 and 26 or a sequence having at least 90% sequence identity with one of these consensus sequences. A SAD-homology domain may also comprise two or all three of the consensus sequences of SEQ ID NOs: 19, 25 and 26 or a sequence having at least 90% sequence identity with one, two or all three of the consensus sequences. SAD-homology domains can be identified by using standard sequence search and alignment tools.

A “consensus sequence” in the context of the present disclosure refers to a sequence of nucleotide or amino acid residues, which represents the most frequent residues for each position in an alignment of sequences. The consensus sequences set forth in SEQ ID NOs: 19, 25 and 26 were the highest conserved sequences, i.e. having the highest frequency of one specific residue at each position, found in an alignment of SAD1/SAD1 sequences thus representing common identifying motifs having a common functional effect in the context of the relevant SAD1/SAD2 protein when expressed.

A “functional mutation” in the context of the present invention has an effect on the expression of the target amino acid sequence. “Functional” implies that a mutation inserted into a nucleic acid sequence has an effect on the amount of correctly transcribed and/or translated product so that the resulting protein is not, to a lesser extent, or is only present in modified form in a cell in comparison to a cell comprising no functional mutation in the respective gene and being able to express the functional wild-type protein. Thus, a functional mutation can be a loss-of-function mutation, i.e. when the expression product is not functional due to its modified, e.g. truncated, form. In particular, a functional mutation results in a decreased or abolished expression of the target sequence meaning that the target sequence is not expressed at all or is expressed in a lesser amount in a cell compared to a cell comprising no functional mutation in the respective sequence. A functional mutation can be a point mutation, and insertion, deletion or substitution of one or more nucleotides, a knock-out or knock down, a stop codon insertion or a frame-shift mutation. A functional mutation may also be present in a regulatory sequence and thus affect the expression of the target sequence.

“Expression” of a gene in the context of the present invention covers all steps involved in the conversion of the information contained in a gene or nucleic acid molecule, into a “gene product” or “expression product”. A “gene product” or “expression product” can be the direct transcriptional product of a gene or nucleic acid molecule (e.g., mRNA, tRNA, rRNA, antisense RNA, ribozyme, structural RNA or any other type of RNA) or a protein produced by translation of an mRNA. The term expression thus covers transcription, including its regulation, initiation and termination as well as translation into nucleic acid sequence.

A “loss of expression” functional mutation in a protein in the context of the present invention refers to a mutation in a protein that causes loss of its native or wild-type function. Said terms thus refers to a mutation in a polynucleotide encoding a protein that causes loss-of-function of said protein. A protein may still be produced from the polynucleotide comprising the loss of expression mutation, but the protein can no longer perform its function or cannot perform its function effectively, for example because a specific point mutation was introduced into the active site of the SAD-homology protein, or because a stop codon has been introduced into the coding sequence resulting in a truncated and thus non-functional SAD-homology protein.

A “loss of transcription mutation” results in loss of expression of the mutated gene or the product encoded by said mutated gene, e.g. by inserting a mutation into the regulatory sequence of the SAD-homology protein encoding sequence, or wherein RNA interference is used, to diminish or abolish transcription of a RNA otherwise encoding a SAD-homology protein. Furthermore, said term comprises a knock-out of a SAD-homology sequence in a plant cell, seed, plant or plant material used as haploid or doubled haploid inducer.

A “regulatory sequence” in the context of the present disclosure refers to a nucleic acid sequence, which controls when and how much expression occurs of a coding region. Regulatory sequences can e.g. represent promoter regions, binding sites for transcription factors, i.e. activators, repressors.

A “knock-out” or “knock-down” of a target gene in the context of the present disclosure refers to a full or partial inactivation of the target gene, respectively. In case of a knock-down, the expression of the target gene is usually reduced, while in case of a “knock-out”, no (functional) gene is expressed at all.

A “haploid plant” or “haploid plant cell” herein refers to a plant or a plant cell having only one set of chromosomes each one not being part of a pair. The number of chromosomes in a single set is called the haploid number, given the symbol n. “Gametes” are haploid cells, of which two combine in fertilization to form a “zygote” with n pairs of chromosomes, i.e. 2n chromosomes in total. Each chromosome pair comprises one chromosome from each gamete, called homologous chromosomes. Cells and organisms with pairs of homologous chromosomes are “diploid”.

“Doubled haploid” in the context of the present disclosure refers to a genotype formed when haploid cells undergo chromosome doubling. Doubled haploids may be produced spontaneously or by chromosome doubling from haploid cells, organisms or material Therefore, doubled haploids are homozygous. For polyploid cell (2n=3×, 4×, 5, 6×, or more), the corresponding polyhaploids may contain more than one copy of the same haploid genome.

A “plant having activity of a haploid inducer” or a “haploid inducer” or a “haploid inducer line” in the context of the present invention is a plant or plant line, which was genetically modified to have the capability to produce haploid offspring in at least 0.1%, at least 0.2%, 0.3%, 0.4%, 0.5%, 0.6%, 0.7%, 0.8%, 0.9%, preferably at least 1.0%, preferably at least 2.0%, least 3.0%, at least 4.0%, at least 5.0%, at least 6.0%, at least 7.0%, at least 8.0%, at least 9.0%, at least 10.0%, at least 11.0%, at least 12.0%, at least 13.0%, at least 14.0%, at least 15.0% of cases when combined in fertilization with a wild-type plant. Since the chromosomes of the haploid inducer are eliminated, the resulting haploid progeny only comprises genetic material of the wild-type parent.

A “plant having activity of a doubled haploid inducer” or a “doubled haploid inducer” or a “doubled haploid inducer line” in the context of the present invention is a plant or plant line, which was genetically modified to have the capability to produce doubled haploid offspring in at least 0.1%, at least 0.2%, 0.3%, 0.4%, 0.5%, 0.6%, 0.7%, 0.8%, 0.9%, preferably at least 1.0%, preferably at least 2.0%, least 3.0%, at least 4.0%, at least 5.0%, at least 6.0%, at least 7.0%, at least 8.0%, at least 9.0%, at least 10.0%, at least 11.0%, at least 12.0%, at least 13.0%, at least 14.0%, at least 15.0% of cases when combined in fertilization with a wild-type plant. The chromosomes of the doubled haploid inducer are eliminated and the resulting doubled haploid progeny only comprises genetic material of the wild-type parent. The doubled haploid progeny is formed spontaneously after fertilization and no chromosome doubling step is necessary.

A nucleic acid sequence that is “endogenous” to a cell or organism refers to a nucleic acid molecule that naturally occurs in the genome of this cell or organism. On the other hand, a nucleic acid molecule that is “exogenous” or to a cell or organism or a “transgene” refers to a nucleic acid molecule that does not naturally occur in this cell or organism but has been introduced by a transgenic event. The term transgenic event shall expressly also include at least one targeted mutation introduced into at least one allele of an endogenous gene e.g. introduced by means of gene editing.

A “gamete” in the context of the present disclosure is a haploid cell that fuses with another haploid cell in fertilization during sexual reproduction. Plants, which reproduce sexually, produce male and female gametes. A haploid inducer or doubled haploid inducer activity may be linked to a male or female gamete or it may be independent of sex. Thus, a plant can be a “paternal and/or maternal” haploid or doubled haploid inducer depending on whether contacting a male or female gamete, or either, with a wild-type gamete results in (doubled) haploid progeny.

By “contacting a first and a second gamete”, a zygote is generated. However, if one gamete is derived from a plant having the activity of a haploid inducer or a doubled haploid inducer, while the other one is derived from a wild-type plant, sexual crossing, i.e. the combination of the two genomes into a diploid genome with two sets of chromosomes, is suppressed in some cases, resulting in a haploid or doubled haploid F1 zygote. This is due to chromosome elimination of the (doubled) haploid inducer and the conservation of the genome of the wild-type parent. In contrast, by sexual crossing, genes of both parents are mixed by homologous recombination in order to obtain new genetic combinations and traits. Obtaining a haploid or doubled haploid cell from the F1 zygote by elimination of the chromosomes of the haploid inducer or the doubled haploid inducer therefore does not occur by a natural process but is the result of genetic engineering of an artificial haploid inducer or doubled haploid inducer plant.

A “not naturally occurring” gamete refers to an artificial gamete, which does not occur in nature but has been genetically modified, in particular, to have the activity of a (doubled) haploid inducer.

Whenever the present disclosure relates to the percentage of identity of nucleic acid or amino acid sequences to each other these values define those values as obtained by using the EMBOSS Water Pairwise Sequence Alignments (nucleotide) program (www.ebi.ac.uk/Tools/psa/emboss_water/nucleotide.html) nucleic acids or the EMBOSS Water Pairwise Sequence Alignments (protein) program (www.ebi.ac.uk/Tools/psa/emboss_water/) for amino acid sequences. Alignments or sequence comparisons as used herein refer to an alignment over the whole length of two sequences compared to each other. Those tools provided by the European Molecular Biology Laboratory (EMBL) European Bioinformatics Institute (EBI) for local sequence alignments use a modified Smith-Waterman algorithm (see www.ebi.ac.uk/Tools/psa/ and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5. The skilled person is well aware of the fact that, for example, a sequence encoding a protein can be “codon-optimized” if the respective sequence is to be used in another organism in comparison to the original organism a molecule originates from.

In the context of the present invention, the sequence identity is to be determined with respect to the full length of the respective sequence given under a SEQ ID NO.

Sequence alignments can be conducted, for example, using the tool multiple sequence alignment Clustal Omega (EMBL-EBI; https://www.ebi.ac.uk/Tools/msa/clustalo/) using the default parameters. The default parameters are: Output format (ClustalW with character counts), dealign input sequences (no), mbed-like clustering guide-tree (yes), mbed-like clustering iteration (yes), number of combined iterations (default(0)),max guide tree iterations(default),max hmm iterations(default), order(aligned). The results were displayed, using the programme JalView (Waterhouse, A. M., Procter, J. B., Martin, D. M. A, Clamp, M. and Barton, G. J. (2009)) with shown consensus histogram and conservation histogram.

### DETAILED DESCRIPTION

The present invention relates to several aspects, which provide means and methods to achieve haploid induction or even doubled haploid induction in plant breeding. In particular, the present invention identifies a new target gene, which was not previously associated with haploid induction, and which—by functional mutation—is surprisingly able to not only facilitate haploid induction but, in a significant amount of cases, spontaneous doubled haploid progeny is formed. This obviates the need of a chromosome doubling step in order to obtain homozygous fertile offspring for further breeding.

In a first aspect, the present invention relates to the use of a plant seed, plant cell or part of a plant comprising a nucleotide sequence encoding an amino acid sequence comprising a SAD-homology domain, wherein the SAD-homology domain comprises at least one functional mutation, wherein the at least one functional mutation results in a decreased or abolished expression of the amino acid sequence comprising a SAD-homology domain in comparison to the cognate wild-type amino acid sequence, wherein the functional mutation affects the expression of a SAD-homology domain consensus sequence as set forth in any one of SEQ ID NOs: 19, 25 and 26, or an amino acid sequence having at least 90%, 91%, 92%, 93%, 94%, 95%, 96%, 97%, 98%, or 99% sequence identity to the sequence set forth in any one of SEQ ID NOs: 19, 25 and 26 for the induction of a haploid or a doubled haploid plant.

SAD proteins have not previously been associated with haploid induction. In the context of the present invention, it was surprisingly found out that by introducing a functional mutation within a SAD-homology domain in a plant, significant haploid induction rates can be achieved. Even more surprisingly, in a significant amount of cases, doubled haploid progeny is formed spontaneously, which can be used in further breeding without the need to perform a chromosome doubling step using e.g. chemical treatment with colchicine. SAD proteins comprise a region comprising a Cse1 domain. This region ranges from position 304 to position 467 referenced to the Arabidopsis thaliana amino acid sequence (SEQ ID NO: 2). This region is higher conserved than the complete sequence (see Table 1 below). Within this region there is between position 351 and 389 a sequence which is almost identical between all SAD targets identified so far.

TABLE 1
SAD2 protein sequence identities and similarities
compared to Arabidopsis thaliana reference
amino acid sequence of SEQ ID NO: 2

| | Complete protein | | region comprising | |
|---|---|---|---|---|
| | sequence | | Cse-1 domain | |
| | Identity | Similarity | Identity | Similarity |
| B. vulgaris ssp. vulgaris | 75.2% | 87.6% | 81.7% | 91.5% |
| B. napus A genome | 90.5% | 94.5% | 88.4% | 94.5% |
| B. napus C genome | 90.0% | 94.3% | 87.8% | 94.5% |
| B. oleracea | 90.2% | 94.3% | 89.0% | 95.1% |
| C. annuum | 76.5% | 89.0% | 81.1% | 90.9% |
| C. sativus | 74.7% | 88.2% | 82.3% | 91.5% |
| G. max | 72.6% | 85.6% | 80.5% | 90.9% |
| H. annuus homolog 1 | 74.4% | 86.6% | 79.3% | 89.0% |
| H. annuus homolog 2 | 74.1% | 86.7% | 79.9% | 88.4% |
| H. annuus homolog 3 | 74.2% | 86.7% | 76.8% | 88.4% |
| S. lycopersicum | 75.6% | 87.9% | 80.5% | 91.5% |
| S. bicolor | 70.2% | 84.8% | 74.4% | 90.2% |
| Z. mays | 70.2% | 85.6% | 73.2% | 90.2% |

As indicated above, a Cse1 domain was described to be essential for the function of SAD1/SAD2. However, Cse1 domains are widely distributed over proteins in different taxa fulfilling different functions. A targeted analysis to identify a possible contribution of selected positions within a Cse1 domain in SAD1/SAD2 proteins in the context of the herein observed and studied effect of (doubled) haploid induction has not been conducted so far. Therefore, three highly conserved consensus sequences have been identified within the Cse1 domain (SEQ ID NOs: 19, 25 and 26), which represent core regions of interest for the effects studied herein and which can serve as basis for transforming the relevant teaching to other SAD1/SAD2 homologous sequences in various target plants.

Notably, in SEQ ID NO: 19, the positions shown as Xaa may be any naturally occurring amino acid.

In SEQ ID NO: 25, Xaa can be any naturally occurring amino acid. In a preferred embodiment, Xaa at position (6) can be any naturally occurring amino acid selected from the group of amino acids containing carboxylic chains, for example glutamic acid and aspartic acid. In another preferred embodiment, Xaa at position (11) can be any naturally occurring amino acid selected from the group consisting of amino acids with aliphatic side chains, for example valine, leucine and isoleucine. In yet another preferred embodiment, Xaa at position (19) can be any naturally occurring amino acid selected from the group of amino acids with aliphatic hydroxyl side chains and cysteine. In another preferred embodiment, Xaa at position (21) can be any naturally occurring amino acid selected from the group of small amino acids and amino acids with aliphatic hydroxyl side chains. In yet another preferred embodiment, Xaa at position (71) and Xaa at position (73) can be any naturally occurring amino acid selected from the group of aromatic amino acids, for example phenylalanine and tyrosine. In another preferred embodiment, Xaa at position (78) can be any naturally occurring amino acid selected from the group consisting of amino acids with aliphatic hydroxyl side chains and carboxamide side chains, for example asparagine and serine and Xaa at position (81) can be any naturally occurring amino acid selected from the group consisting of small amino acids and amino acids with hydroxyl side chains, for example alanine and threonine.

It was further deduced that in SEQ ID NO: 26, Xaa can be any naturally occurring amino acid. In a preferred embodiment, Xaa at position (6) can be any naturally occurring amino acid selected from the group consisting of amino acids with aliphatic side chains, for example valine, leucine and isoleucine. In another preferred embodiment, Xaa at position (13) can be any naturally occurring amino acid selected from the group consisting of amino acids with aliphatic side chains and proline. In yet another preferred embodiment, Xaa at position (14) can be any naturally occurring amino acid selected from the group of amino acids with carboxylic side chains, for example glutamic acid and aspartic acid. In another preferred embodiment, Xaa at position (20) can be any naturally occurring amino acid selected from the group of small amino acids, for example glycine and alanine. In yet another preferred embodiment, Xaa at position (28) can be any naturally occurring amino acid selected from the group consisting of amino acids with aliphatic side chains. In another preferred embodiment, Xaa at position (37) can be any naturally occurring amino acid selected from the group consisting of amino acids with aliphatic hydroxyl side chains. In yet another preferred embodiment, Xaa at position (40) can be any naturally amino acid selected from the group consisting of small amino acids.

Based on the various embodiments and aspects disclosed herein, comparable consensus sequences can be identified in a plant germplasm of interest. The relevant information on the genotype as present in a germplasm of interest can then be used to either introduce a targeted mutation or a transgene, preferably in all relevant alleles, to obtain a plant material suitable as haploid or doubled haploid inducer as further detailed herein below for subsequent breeding efforts to rapidly speed up product development cycles for obtaining plants carrying agronomically favorable traits.

The mechanism of doubled haploid induction remains to be elucidated. However, it is assumed that it is related to the disturbed function of SAD2 as β-importin (Zhao et al. (2007). SAD2, an importin β-like protein, is required for UV-B response Arabidopsis by mediating MYB4 nuclear trafficking. The Plant Cell, 19(11), 3805-3818.) during embryogenesis and reduced stress tolerance (Verslues et al., 2006) or disturbed function of micro RNA pathway (Wang et al., (2011). An importin β protein negatively regulates microRNA activity in Arabidopsis. The Plant Cell, 23(10), 3565-3576. In the Arabidopsis genome, second homologs of SAD2 gene have been identified, designated as a SAD1 gene (At3g59020), which can be used for dihaploid production too.

In a preferred embodiment of the use, all alleles and homologs encoding a SAD-homology domain present in the plant carry functional mutations as described above. Thus, in a preferred embodiment, the plant is homozygous for the functional mutation.

A functional mutation in the SAD-homology domain as required for (doubled) haploid induction may be a point mutation but it may also disrupt the SAD-homology domain e.g. by insertion or deletion of multiple nucleotides so that in the plant used according to the invention, the consensus sequences defined above may no longer be present. However, before the introduction of the functional mutation, at least one of the consensus sequences must have been present to define a SAD-homology domain. SAD-homology domains can be identified by sequence search and alignment. As described in detail below, several SAD-homology domains have been found in different crop plants as presented by SEQ ID NOs: 1 to 4, 6 to 18 and 20 to 24. Using the same methods as described herein, a skilled person can identify more SAD-homology domains accordingly.

In one embodiment, the nucleotide or amino acid sequence comprising a SAD-homology domain is selected from a sequence as set forth in any one of SEQ ID NOs: 1 to 4, 6 to 18 and 20 to 24, or a nucleotide or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in any one of SEQ ID Nos: 1 to 4, 6 to 18 and 20 to 24.

A functional mutation has an effect on the amount of correctly transcribed and/or translated product so that the resulting protein is not present, to a lesser extent, or is only present in modified form in a cell in comparison to a cell comprising no functional mutation in the respective gene and being able to express the functional wild-type protein.

In one embodiment, the functional mutation is selected from a point mutation in the coding sequence, or in a regulatory sequence, a frameshift mutation, an insertion mutation, or a knock-out or knock-down of/in a nucleic acid sequence encoding the sequence comprising a SAD-homology domain or a regulatory sequence thereof, wherein the functional mutation results in a loss of expression or loss of transcription.

A functional mutation can e.g. be a point mutation, an insertion, deletion or substitution of one or more nucleotides in an active site of the protein, a knock-out or knock-down, a stop codon insertion or a frame-shift mutation. A functional mutation may also be present in a regulatory sequence and thus affect the expression of the SAD-homology domain consensus sequence. A skilled person is aware of a number of techniques for the introduction of such mutations. The mutations can e.g. be introduced by random mutagenesis, in particular chemical mutagenesis, preferably via EMS (ethylmethane sulfonate)-induced or ENU (N-ethyl-N-nitrosourea)-induced TILLING or by targeted mutagenesis, preferably by means of meganucleases, Zinc Finger nucleases, TALENs or CRISPR/Cas such as CRISPR/Cas9 or CRISPR/Cpf1, or by means of base editor systems. For a site-directed modification, genome editing tools, i.e. site-specific effectors such as nucleases, nickases, recombinases, transposases, base editors are available. These effectors have the capacity to introduce a single- or double-strand cleavage into a genomic target site, or have the capacity to introduce a targeted modification, including a point mutation, an insertion, or a deletion, into a genomic target site of interest. A double strand break can then repaired by non-homologous end-joining or homology-directed repair, e.g. using a repair template. In a preferred embodiment, T-DNA insertion by Agrobacterium is used to introduce the mutation.

Gametes of plants carrying an above described mutation provide haploid induction rates of up to 15% when forming a zygote with a gamete expressing the wild-type SAD-homology domain. The genome of the plant carrying the mutation is eliminated so that the haploid progeny exclusively carries genomic material from the wild-type parent.

Preferred is an embodiment of the use described above, wherein generating a zygote from the plant, seed, or plant cell and a wild-type plant or a plant expressing a wild-type SAD-homology domain comprising amino acid sequence yields at least 0.5%, preferably at least 1.0%, at least 2.0%; at least 3.0%, at least 4.0% at least 5.0%, at least 6.0%, at least 7.0% at least 8.0%, at least 9.0%, at least 10.0%, at least 11.0%, at least 12.0%, at least 13.0%, at least 14.0% or at least 15.0% haploid progeny.

It is particularly surprising that, in a significant amount of cases, when a gamete of plants carrying an above described mutation forms a zygote with a gamete expressing the wild-type SAD-homology domain, doubled haploid offspring is formed directly, which is homozygous for the wild-type genome.

In a preferred embodiment of the use described above, generating a zygote from the plant, seed, or plant cell and a wild-type plant or a plant expressing a wild-type SAD-homology domain comprising amino acid sequence yields at least 0.5%, preferably at least 1.0%, at least 2.0%; at least 3.0%, at least 4.0% at least 5.0%, at least 6.0%, at least 7.0%, at least 8.0%, at least 9.0%, at least 10.0%, at least 11.0%, at least 12.0%, at least 13.0%, at least 14.0% or at least 15.0% doubled haploid progeny.

In some cases, a mixture of haploid and doubled haploid offspring is obtained. Surprising is in particular the large portion of spontaneously formed doubled haploid progeny.

The gamete of the plant carrying an above described mutation may be the male (i.e. pollen) or female gamete (i.e. egg cell/ovule) in forming a zygote to generate haploid or doubled haploid progeny. Thus, the haploid or doubled haploid inducer activity may be paternal or maternal.

In one embodiment of the use described above, the plant, seed, or plant cell is a paternal and/or maternal haploid or doubled haploid inducer.

In a preferred embodiment, the gamete from the plant, seed, or plant cell carrying the functional mutation in the SAD-homology domain is a male gamete and the plant, seed, or plant cell is therefore a paternal haploid or doubled haploid inducer.

In one embodiment of the use described above, the nucleotide and/or amino acid sequence encoding or comprising the SAD-homology domain comprises at least one endogenous gene or at least one transgene.

It is possible to obtain the haploid or doubled haploid inducer activity by either introducing the at least one mutation in an endogenous nucleotide sequence or introducing the sequence as a transgene. Thus, a completely transgene-free approach may be chosen or a transgenic plant according to the invention may be provided. This can be effected by inserting a desired mutation based on the findings on conserved SAD1/SAD2 consensus sequences as identified herein, for example, by using a transient genome editing technique to introduce a desired mutation into at least one, preferably all, alleles of a sad1 and/or sad2 gene of interest in a germplasm of a plant of interest.

When a transgenic plant having the activity of a (doubled) haploid inducer is used in a method for obtaining a (doubled) haploid plant as described herein, the genome of the haploid inducer is eliminated resulting in a transgene-free (doubled) haploid plant. Therefore, non-transgenic haploid plants can be provided, which is advantageous due to the regulatory limitations imposed on transgenic plants.

The plant seed, plant cell or part of a plant used according to the invention in any of the embodiments described above may be derived from any agronomically important crop plants carrying a SAD-homology domain.

In particular, the plant seed, plant cell or part of a plant may be derived from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus ca rota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and/or Allium tuberosum.

In a further embodiment of the use described above, the cell or part of the plant is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

According to another aspect, the present invention relates to a haploid plant obtained by contacting a first gamete from a plant as described above with a second a gamete from a plant expressing an amino acid sequence comprising a wild-type SAD-homology domain to generate a zygote.

Haploid offspring is obtained in a significant amount of cases when a gamete from a plant carrying a functional mutation in the SAD-homology domain is contacted with a gamete from a plant expressing an amino acid sequence comprising a wild-type SAD-homology domain to generate a zygote. The genome of the plant carrying the functional mutation has been eliminated in the haploid offspring, so that the haploid plant only contains genetic material from the plant expressing an amino acid sequence comprising a wild-type SAD-homology domain. The plant is therefore not genetically modified. Subsequently, the haploid plant can be subjected to a chromosome doubling, e.g. by colchicine treatment, to generate a doubled haploid plant being homozygous for a desired trait. This plant can be further used in breeding protocols.

According to a further aspect, the present invention relates to a doubled haploid plant obtained by converting the haploid plant described above into a doubled haploid plant, preferably a spontaneous doubled haploid plant directly obtained by contacting a first gamete from a plant as defined in any of the embodiments described above with a second a gamete from a plant expressing an amino acid sequence comprising a wild-type SAD-homology domain.

A homozygous doubled haploid plant can be obtained by subjecting the haploid plant described above to chromosome doubling, e.g. by colchicine treatment. Advantageously, however, doubled haploid plants are also formed spontaneously obviating the chromosome doubling step and the associated additional effort and handling of toxic substances. The doubled haploid plant exclusively comprises genetic material from the parent expressing the wild-type SAD-homology domain as the genome of the SAD-homology domain mutant has been eliminated. The doubled haploid plant is therefore homozygous for desired traits of the wild-type parent, not genetically modified and can directly be used in further breeding protocols.

The haploid or doubled haploid plant according to the aspects described above may be selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and/or Allium tuberosum.

According to a further aspect, the present invention also relates to a nucleic acid sequence encoding a consensus sequence as set forth in any one of SEQ ID NOs: 19, 25 and 26, or a sequence having at least 90%, 91%, 92%, 93%, 94%, 95%, 96%, 97%, 98%, or 99% sequence identity thereto, or a nucleotide sequence or an amino acid sequence as set forth in any one of SEQ ID NOs: 1 to 4, 6 to 18 and 20 to 24, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

The consensus sequences of SEQ ID NOs: 19, 25 and 26, represent highly conserved motifs identified in the SAD-homology domains of different plants represented by SEQ ID NOs: 1 to 4, 6 to 18 and 20 to 24. They therefore represent the targets for a functional mutation resulting in the haploid or doubled haploid induction activity according to the present invention. SAD-homology domains, e.g. as represented by SEQ ID NOs: 1 to 4, 6 to 18 and 20 to 24, have not previously been associated with haploid induction. Thus, it was surprising that a functional mutation in this domain is capable of conferring to a plant the activity of a haploid inducer. Even more surprising is the fact that such mutations also confer to a plant the activity of a doubled haploid inducer.

Haploid induction for obtaining plant cells containing only the chromosome set found after meiosis in male (sperm cells) or female (egg cells) gametes is a relevant process in plant breeding. The numerous methods to obtain haploid plants for different cultivars of commercial relevance can be roughly classified into two categories. Firstly, in vitro methods are based on the culture of haploid cells and their differentiation into haploid embryos and ultimately haploid plants. Both male (microspores or pollen) and female haploid cells (megaspores or ovules) are used, depending on the responsiveness of the cells in a given species. Secondly, in situ methods make use of particular pollination techniques using irradiated pollen, inter-specific crosses or so-called ‘inducer lines’. The skilled person is well aware of suitable techniques for in vitro or in vivo haploid induction in relevant plants, and particular crop, species like in Zea mays, see for example, Prigge and Melchinger, Production of Haploids and Doubled Haploids in Maize, Plant Cell Culture Protocols (2012).

As it is known to the skilled person, doubled haploids can be created when a haploid (n) plant undergoes genome doubling due to the exposure to a doubling agent or mitotic inhibitor, the most common chromosome doubling agent being colchicine. This doubling results in genetically uniform diploid offspring with two identical genomes (2n). Further chromosome doubling techniques are well-known to the skilled person for various crops.

For Zea mays, a common method of producing haploid plants is in vivo maternal haploid induction requiring a specific inducer genotype. When an inducer is crossed to a maize donor plant, progeny will segregate into diploid (2n) and haploid (n) classes. Further methods are disclosed in, for example, Vanous et al., Generation of maize (Zea may) doubled haploids via traditional methods, Current Protocols in n Plant Biology: 147-157, 2017. Various protocols are available to the skilled person for doubled haploid generation for other relevant plant species.

According to yet another aspect, the present invention relates to a method of generating a haploid or doubled haploid plant cell, comprising the steps of:
a) providing a first gamete, preferably a not naturally occurring first gamete, from a plant having the activity of a haploid or doubled haploid inducer as defined in any of the embodiments above;
b) contacting the first gamete from step a), preferably a male gamete, with a second gamete of a wild-type plant, or of a plant comprising a wild-type SAD-homology domain comprising amino acid sequence or a sequence encoding the same to generate a F1 zygote;
c1) obtaining a haploid cell by elimination of the chromosomes of the plant having the activity of a haploid inducer from the F1 zygote, or
c2) directly obtaining a doubled haploid F1 zygote cell.

In step a) of the method, a first gamete is provided, which is derived from a plant comprising a nucleotide sequence encoding an amino acid sequence comprising a SAD-homology domain, wherein the SAD-homology domain comprises at least one functional mutation.

The first gamete is preferably a not naturally occurring gamete, because it has been genetically modified to have haploid or doubled haploid inducer activity. The plant from which the gamete is derived has been obtained by introducing at least one functional mutation, which results in a decreased or abolished expression of the amino acid sequence comprising a SAD-homology domain in comparison to the cognate wild-type amino acid sequence. The functional mutation affects the expression of a SAD-homology domain consensus sequence as set forth in any one of SEQ ID NOs: 19, 25 and 26, or an amino acid sequence having at least 90%, 91%, 92%, 93%, 94%, 95%, 96%, 97%, 98%, or 99% sequence identity to the sequence set forth in any one of SEQ ID NOs: 19, 25 and 26.

In an embodiment of the method described above, the plant from which the first gamete is derived, is the female parent, and the plant from which the second gamete is derived, is the male parent, or the plant from which the first gamete is derived is the male parent, and the plant from which the second gamete is derived, is the female parent. Preferably, the plant from which the first gamete is derived, is the male parent, and the plant from which the second gamete derived, is the female parent.

Contacting the first gamete with the second gamete in step b) results in the formation of an F1 zygote, from which in a certain amount of cases, the chromosomes of the first gamete are eliminated and no sexual crossing of genomes occurs. Furthermore, in a certain amount of cases, the chromosomes of the second gamete double spontaneously. Thus, haploid and/or doubled haploid offspring is obtained in step c1) and/or c2). The haploid and/or doubled haploid offspring is the result of genetic engineering of an artificial haploid or doubled haploid inducer plant and the step of selectively contacting a gamete from such a plant with a gamete from a wild-type plant or a plant expressing wild-type SAD-homology domain. The haploid or doubled haploid progeny is therefore not formed by processes occurring in nature. The haploid and/or doubled haploid cell obtained in step c1) or c2) only comprises genetic material from the second gamete and thus does not comprise the genetic modification of the first gamete, which confers the haploid or doubled haploid inducer activity.

In one embodiment, the haploid cell obtained in step c1) may be subjected to a step of chromosome doubling, e.g. by colchicine treatment.

The doubled haploid cell obtained in step c2) is homozygous and can directly be propagated.

In one embodiment of the method described above, the amino acid sequence comprising a SAD-homology domain is selected from an amino acid sequence as set forth in any one of SEQ ID NOs: 1 to 4, 6 to 18 and 20 to 24, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in any one of SEQ ID NOs: 1 to 4, 6 to 18 and 20 to 24.

The haploid or doubled haploid plant obtained in step c1) and/or c2) may be selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus ca rota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and/or Allium tuberosum.

In one embodiment of the method described above, the functional mutation is selected from a point mutation in the coding sequence, or in a regulatory sequence, a frameshift mutation, an insertion mutation, or a knock-out or knock-down of/in a nucleic acid sequence encoding the sequence comprising a SAD-homology domain or a regulatory sequence thereof, wherein the functional mutation results in a loss of expression or loss of transcription.

In another embodiment of the method described above, contacting the first and second gamete in step b) yields at least 0.5%, preferably at least 1.0%, at least 2.0%; at least 3.0%, at least 4.0% at least 5.0%, at least 6.0%, at least 7.0% at least 8.0%, at least 9.0%, at least 10.0%, at least 11.0%, at least 12.0%, at least 13.0%, at least 14.0% or at least 15.0% haploid progeny in step c1) and/or yields at least 0.5%, preferably at least 1.0%, at least 2.0%; at least 3.0%, at least 4.0% at least 5.0%, at least 6.0%, at least 7.0%, at least 8.0%, at least 9.0%, at least 10.0%, at least 11.0%, at least 12.0%, at least 13.0%, at least 14.0% or at least 15.0% doubled haploid progeny in step c2).

In one embodiment, the method described above comprises in addition to steps a) to c) the following steps:
d1) growing the haploid cell under conditions to obtain a haploid plant or a part thereof; and
e1) obtaining a haploid plant or part thereof; or
d2) growing the doubled haploid cell under conditions to obtain a doubled haploid plant or a part thereof; and
e2) obtaining a doubled haploid plant or part thereof.

The method described above produces doubled haploid plant lines, which can be used in breeding for efficiently developing desirable traits. Doubled haploid lines can also be used as parents in hybrid production as explained in further detail below.

In one aspect, the present invention relates to a method of directly generating a doubled haploid plant cell comprising the steps of:
a) providing a first gamete, preferably a not naturally occurring first gamete, from a plant having the activity of a doubled haploid inducer as defined in any of the embodiments above,
b) contacting the first gamete from step a), preferably a male gamete, with a second gamete of a wild-type plant, or of a plant comprising a wild-type SAD-homology domain comprising amino acid sequence or a sequence encoding the same to generate a F1 zygote;
c) directly obtaining a doubled haploid cell as F1 zygote.

Surprisingly, in a certain amount of cases, the chromosomes of the second gamete double spontaneously when the chromosomes of the first gamete are eliminated. The doubled haploid cell obtained in step c) only comprises genetic material from the second gamete and thus does not comprise the genetic modification of the first gamete, which confers the haploid or doubled haploid inducing activity. The doubled haploid cell obtained in step c) is homozygous and can directly be propagated.

The first gamete is preferably a not naturally occurring gamete, because it has been genetically modified to have haploid or doubled haploid inducer activity. The plant from which the gamete is derived has been obtained by introducing at least one functional mutation, which results in a decreased or abolished expression of the amino acid sequence comprising a SAD-homology domain in comparison to the cognate wild-type amino acid sequence. The functional mutation affects the expression of a SAD-homology domain consensus sequence as set forth in any one of SEQ ID NOs: 19, 25 and 26, or an amino acid sequence having at least 90%, 91%, 92%, 93%, 94%, 95%, 96%, 97%, 98%, or 99% sequence identity to the sequence set forth in any one of SEQ ID NOs: 19, 25 and 26.

In an embodiment of the method described above, the plant from which the first gamete is derived, is the female parent, and the plant from which the second gamete is derived, is the male parent, or the plant from which the first gamete is derived, is the male parent, and the plant from which the second gamete is derived, is the female parent. Preferably, the plant from which the first gamete is derived, is the male parent, and the plant from which the second gamete is derived, is the female parent.

In one embodiment of the method described above, the amino acid sequence comprising a SAD-homology domain is selected from an amino acid sequence as set forth in any one of SEQ ID NOs: 1 to 4, 6 to 18 and 20 to 24, or an amino acid sequence having at least 75%, 76%, 77%, 78%, 79%, 80%, 8M), 82%, 83%, 84%, 85%, 86%, 87%, 88%, 89%, 90%, 91%, 92%, 93%, 94%, 95%, 96%, 97%, 98%, or 99% sequence identity to the sequence set forth in any one of SEQ ID NOs: 1 to 4, 6 to 18 and 20 to 24.

The doubled haploid plant obtained in step c) may be selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and/or Allium tuberosum.

In one embodiment of the method described above, the functional mutation is selected from a point mutation in the coding sequence, or in a regulatory sequence, a frameshift mutation, an insertion mutation, or a knock-out or knock-down of/in a nucleic acid sequence encoding the sequence comprising a SAD-homology domain or a regulatory sequence thereof, wherein the functional mutation results in a loss of expression or loss of transcription.

In another embodiment of the method described above, contacting the first and second gamete in step b) yields at least 0.5%, preferably at least 1.0%, at least 2.0%; at least 3.0%, at least 4.0% at least 5.0%, at least 6.0%, at least 7.0%, at least 8.0%, at least 9.0%, at least 10.0%, at least 11.0%, at least 12.0%, at least 13.0%, at least 14.0% or at least 15.0% doubled haploid progeny in step c2).

In one embodiment of the method of generating a doubled haploid plant, plant material or seed described above, the method further comprises, in addition to steps a) to c) according to claim 13, the following steps:
d) growing the doubled haploid cell; and
e) obtaining a doubled haploid plant, part thereof, or seed.

The methods described herein produce (doubled) haploid plant lines, which are highly useful in breeding protocols for efficiently developing desirable traits. Doubled haploid lines can be directly used as parents in hybrid production. In hybrid breeding, two different inbred lines are crossed with the aim of obtaining superior traits compared to either of the parents. The inbred lines are genetically uniform due to generations of self-crossing. Using the methods according to the invention, however, genetically uniform parents can be produced in just one generation, even without a chromosome doubling step. This greatly expedites and facilitates the production of hybrid plant lines with superior characteristics.

According to another aspect, the present invention relates to a method for identifying a plant having activity of a haploid inducer or a doubled haploid inducer comprising the step of:
a) providing a population of plants comprising a nucleotide sequence encoding an amino acid sequence comprising a SAD-homology domain;
b) screening the plant population for the presence of at least one functional mutation in the SAD-homology domain, wherein the at least one functional mutation results in a decreased or abolished expression of the amino acid sequence comprising a SAD-homology domain in comparison to the cognate wild-type amino acid sequence, wherein the functional mutation affects the expression of a SAD-homology domain consensus sequence as set forth in any one of SEQ ID NOs: 19, 25 and 26, or an amino acid sequence having at least 90%, 91%, 92%, 93%, 94%, 95%, 96%, 97%, 98%, or 99% sequence identity to the sequence set forth in any one of SEQ ID NOs: 19, 25 and 26; and
c) optionally, obtaining a plant having activity of a haploid or a doubled haploid inducer.

The population of plants provided in step a) can be a naturally occurring population or it can be obtained by performing mutagenesis. For example, T-DNA insertion by Agrobacterium, ethyl methanesulfonate (EMS) mutagenesis or ENU (N-ethyl-N-nitrosourea)-induced TILLING can be used to provide SAD-homology domain mutants conferring haploid or doubled haploid inducer activity.

The screening step b) can be performed by various techniques available to the skilled person. For example, phenotype screening using certain markers, PCR methods involving specific primers and probes, deep sequencing and comparison with wild-type sequences can be employed. The skilled person is well aware of how to design primers to identify mutations in a specific target sequence.

In a preferred embodiment, all alleles and homologs encoding a SAD-homology domain present in the plant identified and optionally obtained in step c) carry functional mutations as described above. Thus, in a preferred embodiment, the plant is homozygous for the functional mutation.

In one embodiment of the method for identifying a plant having activity of a haploid inducer or a doubled haploid inducer described above, the nucleotide or amino acid sequence comprising a SAD-homology domain is selected from a sequence as set forth in any one of SEQ ID NOs: 1 to 4, 6 to 18 and 20 to 24, or a nucleotide or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in any one of SEQ ID Nos: 1 to 4, 6 to 18 and 20 to 24.

In another embodiment of the method for identifying a plant having activity of a haploid inducer or a doubled haploid inducer described above, the functional mutation is selected from a point mutation in the coding sequence, or in a regulatory sequence, a frameshift mutation, an insertion mutation, or a knock-out or knock-down of/in a nucleic acid sequence encoding the sequence comprising a SAD-homology domain or a regulatory sequence thereof, wherein the functional mutation results in a loss of expression or loss of transcription.

In a further embodiment of the method for identifying a plant having activity of a haploid inducer or a doubled haploid inducer described above, generating a zygote from the plant, seed, or plant cell and a wild-type plant or a plant expressing a wild-type SAD-homology domain comprising amino acid sequence yields at least 0.5%, preferably at least 1.0%, at least 2.0%; at least 3.0%, at least 4.0% at least 5.0%, at least 6.0%, at least 7.0%, at least 8.0%, at least 9.0%, at least 10.0%, at least 11.0%, at least 12.0%, at least 13.0%, at least 14.0% or at least 15.0% haploid and/or doubled haploid progeny.

The identified plant, optionally obtained in step c) may be a paternal and/or a maternal haploid or doubled haploid inducer. Preferably, the identified plant is a paternal haploid or doubled haploid inducer.

The plant obtained in step c) can be used for haploid or doubled haploid induction in the context of any embodiment of the uses and methods for generating haploid or doubled haploid plants described above.

A functional mutation in the SAD-homology domain identified in the screening step b) can be introduced in a targeted manner into any plant having a SAD-homology domain to produce a plant having haploid or doubled haploid inducer activity. Methods and tools are available to the skilled person to introduce a targeted mutation in a SAD-homology domain, which results in a decreased or abolished expression of the amino acid sequence comprising a SAD-homology domain in comparison to the cognate wild-type amino acid sequence, resulting in the production of a plant or part of a plant having haploid and/or doubled haploid inducer activity. In particular, a targeted mutation is directed at modifying the expression of any one of the SAD-homology domain consensus sequence as set forth in any one of SEQ ID NOs: 19, 25 and 26. For a site-directed modification of the identified target sequence, genome editing tools such as site-specific effectors, e.g. nucleases, nickases, recombinases, transposases, base editors are available. These effectors can introduce a single- or double-strand cleavage into a genomic target site, or a targeted modification, including a point mutation, an insertion, or a deletion, into a genomic target site of interest. A double strand break can then repaired by non-homologous end-joining or homology-directed repair, e.g. using a repair template. Thus, one a plant having activity of a haploid inducer or a doubled haploid inducer can be produced.

Based on the above disclosure, plant material having the function of a haploid or even doubled haploid inducer can thus be rapidly identified and/or introduced into a germplasm of interest to drastically enhance breeding efforts.

The present invention will now be described in more detail based on the enclosed non-limiting Examples.

### EXAMPLES

### Example 1

T-DNA mutants of Arabidopsis thaliana were analyzed for their ability to induce the generation of haploids in crosses with wild-type. This screening resulted in the selection of a few candidate genes, whereby one of these genes is SAD2 (AT2G31660). It was found that pollination of the trichomeless glabral A. thaliana mutant (g11) with pollen of the SAD2 (Sensitive to ABA and Drought 2) T-DNA insertion mutant (WiscDsLox477-480P7; https://www.arabidopsis.org/servlets/TairObject?type=germplasm&name=WiscDsLox477-480P7) resulted in 3-15% doubled haploid seedlings which only carried the maternal genome. The gl1 mutant had served as a tool for preselecting potential haploid or doubled haploid progeny.

To eliminate potential self-pollination products from double haploids, ecotype-specific PCR-markers were used for genotyping of F1 seedlings (glabral mutant was generated in Ler-0 ecotype and the SAD2 T-DNA mutants in Col-0).

### WiscDsLox477-480P7:

Sequence tagged T-DNA insertion line, generated by vacuum infiltration of Columbia (Col) plants with Agrobacterium tumefaciens vector WiscDs-Lox; Basta herbicide was employed for selection of plants carrying a T-DNA; each T1 transformant has been maintained individually. This line can be utilized as in Ds Launchpad experiments; the T-DNA includes a Ds transposon/launchpad construction so that transposition can be induced by crossing to an Ac line; selection of resulting transposants is by Basta and hygromycin; LoxP sites are also present so that regions between transposed Ds elements and the empty donor site can be subsequently removed by crossing to a Cre line (Woody, Austin-Phillips, Amasino, Krysan: “The WiscDsLox T-DNA collection: an Arabidopsis community resource generated by using an improved high-throughput T-DNA sequencing pipeline”, J. Plant Res., 2007, 120(1): 157-65).

Insertion Flanking Sequence of WiscDsLox477-480P7 is represented by the sequence set forth in SEQ ID NO: 5.

Further suitable mutants were found at http://www.arabidopsis.org/ under the given designations.

### Example 2

### Determination of Consensus Sequences

Originating from our experimental data, other SAD homology proteins in various crop species were searched using the BLAST-N from NCBI at the U.S. National Library of Medicine using an expect threshold of 10, a word size of 28 and Match/Mismatch Scores of 1 and -2 with linear gap costs. The found sequences were taken for pairwise alignment to search for conserved regions. Multi Sequence Alignment by Clustal Omega was done with no dealigned input sequences, an MBED-like clustering guide-tree and a MBED-like clustering iteration. The number of combined iterations, the maximum guide tree iterations and the maximum HMM iterations were set on default. The data thus obtained can be immediately analyzed by MView (version 1.65). From this bioinformatical alignment, consensus sequences can be concluded as all aligned sequences show highly conserved regions. With regard to possible functional motifs and domains the consensus sequences can be determined by selecting these highly conserved regions which show only a small variation in amino acid sequence and an optional conserved functional domain such as a peptide binding site. The deriving consensus sequences from this alignment are SEQ ID NO: 19, SEQ ID NO: 25 and SEQ ID NO: 26. These in silico generated consensus motifs built the basis for additional experimental work.

1. A plant cell or part of a plant comprising a nucleotide sequence encoding an amino acid sequence comprising a SAD-homology domain, wherein the SAD-homology domain comprises at least one functional mutation, wherein the at least one functional mutation results in a decreased or abolished expression of the amino acid sequence comprising a SAD-homology domain in comparison to the cognate wild-type amino acid sequence, wherein the functional mutation affects the expression of a SAD-homology domain consensus sequence as set forth in any one of SEQ ID NOs: 19, 25 and 26, or an amino acid sequence having at least 90%, 91%, 92%, 93%, 94%, 95%, 96%, 97%, 98%, or 99% sequence identity to the sequence set forth in any one of SEQ ID NOs: 19, 25 and 26 for the induction of a haploid or a doubled haploid plant.

2. The plant seed, plant cell or part of the plant according to claim 1, wherein the nucleotide or amino acid sequence comprising a SAD-homology domain is selected from a sequence as set forth in any one of SEQ ID NOs: 1 to 4, 6 to 18 and 20 to 24, or a nucleotide or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in any one of SEQ ID NOs1 to 4, 6 to 18 and 20 to 24.

3. The plant seed, plant cell or part of the plant according to claim 1, wherein the functional mutation is selected from a point mutation in the coding sequence, or in a regulatory sequence, a frameshift mutation, an insertion mutation, or a knock-out or knock-down of/in a nucleic acid sequence encoding the sequence comprising a SAD-homology domain or a regulatory sequence thereof, wherein the functional mutation results in a loss of expression or loss of transcription.

4. The plant seed, plant cell or part of the plant according to claim 1, wherein generating a zygote from the plant, seed, or plant cell and a wild type plant or a plant expressing a wild-type SAD-homology domain comprising amino acid sequence yields at least 0.5%, preferably at least 1.0%, at least 2.0%; at least 3.0%, at least 4.0% at least 5.0%, at least 6.0%, at least 7.0% at least 8.0%, at least 9.0%, at least 10.0%, at least 11.0%, at least 12.0%, at least 13.0%, at least 14.0% or at least 15.0% haploid progeny.

5. The plant seed, plant cell or part of the plant according to claim 1, wherein generating a zygote from the plant, seed, or plant cell and a wild type plant or a plant expressing a wild-type SAD-homology domain comprising amino acid sequence yields at least 0.5%, preferably at least 1.0%, at least 2.0%; at least 3.0%, at least 4.0% at least 5.0%, at least 6.0%, at least 7.0%, at least 8.0%, at least 9.0%, at least 10.0%, at least 11.0%, at least 12.0%, at least 13.0%, at least 14.0% or at least 15.0% doubled haploid progeny.

6. The plant seed, plant cell or part of the plant according to claim 1, wherein the plant, seed, or plant cell is a paternal and/or maternal haploid or doubled haploid inducer.

7. The plant seed, plant cell or part of the plant according to claim 1, wherein the nucleotide and/or amino acid sequence encoding or comprising the SAD-homology domain comprises at least one endogenous gene or at least one transgene.

8. A haploid plant obtained by contacting a first gamete from a plant as defined in claim 1 with a second a gamete from a plant expressing an amino acid sequence comprising a wild-type SAD-homology domain to generate a zygote.

9. A doubled haploid plant obtained by converting the haploid plant according to claim 8 into a doubled haploid plant.

10. A nucleic acid sequence encoding a consensus sequence as defined in claim 1, or a sequence having at least 90%, 91%, 92%, 93%, 94%, 95%, 96%, 97%, 98%, or 99% sequence identity thereto.

11. A method of generating a haploid or doubled haploid plant cell, comprising the steps of:
a) providing a first gamete from a plant having the activity of a haploid or doubled haploid inducer as defined in claim 1;
b) contacting the first gamete from step a), preferably a male gamete, with a second gamete of a wild-type plant, or of a plant comprising a wild-type SAD-homology domain comprising amino acid sequence or a sequence encoding the same to generate a F1 zygote;
c1) obtaining a haploid cell by elimination of the chromosomes of the plant having the activity of a haploid inducer from the F1 zygote, or
c2) directly obtaining a doubled haploid F1 zygote cell.

12. The method of generating a haploid plant according to claim 11, wherein the method comprises, in addition to steps a) to c) the following steps:
d1) growing the haploid cell under conditions to obtain a haploid plant or a part thereof; and
e1) obtaining a haploid plant or part thereof; or
d2) growing the doubled haploid cell under conditions to obtain a doubled haploid plant or a part thereof; and
e2) obtaining a doubled haploid plant or part thereof.

13. A method of directly generating a doubled haploid plant cell comprising the steps of:
a) providing a first gamete from a plant having the activity of a doubled haploid inducer as defined in claim 1,
b) contacting the first gamete from step a), preferably a male gamete, with a second gamete of a wild-type plant, or of a plant comprising a wild-type SAD-homology domain comprising amino acid sequence or a sequence encoding the same to generate a F1 zygote;
c) directly obtaining a doubled haploid cell as F1 zygote.

14. The method of generating a doubled haploid plant, plant material or seed, wherein the method further comprises, in addition to steps a) to c) according to claim 13, the following steps:
d) growing the doubled haploid cell; and
e) obtaining a doubled haploid plant, part thereof, or seed.

15. A method for identifying a plant having activity of a haploid inducer or a doubled haploid inducer comprising the step of:
a) providing a population of plants comprising a nucleotide sequence encoding an amino acid sequence comprising a SAD-homology domain;
b) screening the plant population for the presence of at least one functional mutation in the SAD-homology domain, wherein the at least one functional mutation results in a decreased or abolished expression of the amino acid sequence comprising a SAD-homology domain in comparison to the cognate wild-type amino acid sequence, wherein the functional mutation affects the expression of a SAD-homology domain consensus sequence as set forth in any one of SEQ ID NOs: 19, 25 and 26, or an amino acid sequence having at least 90%, 91%, 92%, 93%, 94%, 95%, 96%, 97%, 98%, or 99% sequence identity to the sequence set forth in any one of SEQ ID NOs: 19, 25 and 26; and
c) optionally, obtaining a plant having activity of a haploid or a doubled haploid inducer.


SEQUENCE LISTING (26 sequences)

SEQ ID NO: 1 - DNA, 3123 bp, Artificial Sequence
  cDNA SAD2 of Arabidopsis thaliana
atggatctgcatagcctcgctttgatcctccgaaccgccgctcttagccctattcccgatgagcgtaaagtttctgagcagcaactcaatcagttggagcacacgccccagcatttggtgaggttattgcagatagccgtggatggaaattgtgacatggcagtgcgtcaaattgctagtattcagttcaaaaatttaattgctaagaactggtcacctgaggattgtggtcctgcagtaaggcagcagcagatatttgagagtgacaaagaattggtgagggacaatattctcgtctatgtcacccaagttccaaccttactcagatcacaacttggagagtctctcaagacaattatttatgctgactacccagaacaatggccacgtcttctggattgggtgaagtacaacttgcaaaatcaacaaatttatggggctttgtttgtgttgcggatactctctagaaaatatgagttcaagtcagatgaggagaggacaccggtttcccgcattgtggaggagacatttcctcaacttttgactatttttaatgggcttatccagataccaaatccgtctctggagatagcagaacttatgaagttgatatgcaaaatattttggtcatccatatatctggagcttccaaggcaattatttgatctaaacgtctttaatgcctggatggttctgttcttgagtgtttcggaacgtcctgttccagttgaaggtcagcctatggatccggaacttagaaaatcttggggctggtggaaggtcaagaagtggacagtgcacattttaaacaggctctatagtcggtttggagatccaaaacttcaaagtccagaaaacaaacctttcgcccaaatgtttcaaaagaattatgcaggcagaattttggaaggccacctaaatttcttgaacacaattcgtgttggaggctatcttcctgacagagttatcaatcttctccttcagtacttaagcaacagcatttcgaagaatagcatgtacaagttgcttctgcctcggctggatgttctgctttttgagattgttttccctctaatgtgctttaatgataatgatcaaaagctttgggaggaagaccctcatgaatatgtgaggaaaggttacaatatcattgaagacttgtacagtccgcgaacagcgtccatggattttgtaaatgagttggttcgaaaacgtgggaaagaaaaccttccgaagtttgttaagtttgtcgttgagatcttcttgagttatgaaaaagctactgtagaggagaagccttatcgccaaaaagatggtgcgatgctggctgttggagctctttgtgacaaattgaagcaaacggatccctataaatctcaactagagctaatgttggtgcaacatatttttcctgatttcaatagtccagttggacatcttagagcaaaggctgcatgggtagctgggcaatatgcacacatcaatttctcagatcagaacaactttcgtaaagcattgcacagtgttgtttcgggactgcgcgatcctgatctccctgttcgtgttgattcagtttttgcattgcgttcatttgttgaggcgtgcaaggatttaaatgagatacgtccaatcctccctcagttacttgatgaatttttcaaactcatgaatgaggtagagaatgaggaccttgtttttactttggagaccatcgtagataagtttggcgaagagatggctccttttgcttttggattatgtcaaaatctggcggctgcgttttggagatgtctaaacacgtcagaagccaatgacgactctgatgacatgggagctttagctgccgttggttgtttgcgtgccataagtacaatccttgaatctgttagcagtctcccgcagctgtttgttgagatagaaccaactatacttccaataatgcagaaaatgttgaccactgatggccaagaggtatttgaagaagttttggagattgcatcatacatgaccttttattcacctagcatttccttggatatatggagtctctggcctttaatggtggaagcattggttgattggggaatcgatttctttccaaatattttggttccaatggacaactttatatcaaggggaacggctcattttctcacttgcaaagagcccgactatcagcaaagtctatataacgttctttcaactcttatgaccgacagaaacatagaagacagtgaaattgagtctgctccaaagcttattgaagttgttttccagaattgcaaagggcaggtggatcagtgggttgaaccatatctgcgtctcactgttgaccggttacaacgggctgagacttcgtatgttaaatctcttcttatacaagtggtggcaaatatgctttactacaatccaggtttgacgcttggtgtattgcataacacaggacttgcttccaaagtctttgacctttggttccagatgttgcagcaaaagagaaaaagtggcctgccagcaaacttcaaaagggaacatgataaaaaggtttgctgcttgggtttgacttcattacttgctctccctgggggtcaattccccgatgaagcgctgcagcgtgttttcagggcaacacttgatcttctagttgcatataagaatcagctagctgaagcagctaaggagacagaagtagattatgaagaagaaatgaatggactccagagtagtgatgatgattatgatgatgatgggtctgatggggagatggatgatacagaggaaggagatgaagcacaaagtgttaaactgcagaagttagctgcacaggcaaaggccttccactatgatgatgatgatgacgacgattctgatgatgactttagtgatgaggacgagtttcagtcacccatcgatgaggtggatgcctttgtattctttgtcgatgcaatcagagttatgcaggcatcagatgcgcagaggtttcagaacctgaaccagtcactggacttcacttaccaggcgattgcaaacggaatagcacagcatgcagagctgagaagagtggagatcgagaaggagaagcaaaagaagttagcagctgcttctactcctgttactgctctatga

SEQ ID NO: 2 - protein, 1040 aa, Arabidopsis thaliana
MDLHSLALILRTAALSPIPDERKVSEQQLNQLEHTPQHLVRLLQIAVDGNCDMAVRQIASIQFKNLIAKNWSPEDCGPAVRQQQIFESDKELVRDNILVYVTQVPTLLRSQLGESLKTIIYADYPEQWPRLLDWVKYNLQNQQIYGALFVLRILSRKYEFKSDEERTPVSRIVEETFPQLLTIFNGLIQIPNPSLEIAELMKLICKIFWSSIYLELPRQLFDLNVFNAWMVLFLSVSERPVPVEGQPMDPELRKSWGWWKVKKWTVHILNRLYSRFGDPKLQSPENKPFAQMFQKNYAGRILEGHLNFLNTIRVGGYLPDRVINLLLQYLSNSISKNSMYKLLLPRLDVLLFEIVFPLMCFNDNDQKLWEEDPHEYVRKGYNIIEDLYSPRTASMDFVNELVRKRGKENLPKFVKFVVEIFLSYEKATVEEKPYRQKDGAMLAVGALCDKLKQTDPYKSQLELMLVQHIFPDFNSPVGHLRAKAAWVAGQYAHINFSDQNNFRKALHSVVSGLRDPDLPVRVDSVFALRSFVEACKDLNEIRPILPQLLDEFFKLMNEVENEDLVFTLETIVDKFGEEMAPFAFGLCQNLAAAFWRCLNTSEANDDSDDMGALAAVGCLRAISTILESVSSLPQLFVEIEPTILPIMQKMLTTDGQEVFEEVLEIASYMTFYSPSISLDIWSLWPLMVEALVDWGIDFFPNILVPMDNFISRGTAHFLTCKEPDYQQSLYNVLSTLMTDRNIEDSEIESAPKLIEVVFQNCKGQVDQWVEPYLRLTVDRLQRAETSYVKSLLIQVVANMLYYNPGLTLGVLHNTGLASKVFDLWFQMLQQKRKSGLPANFKREHDKKVCCLGLTSLLALPGGQFPDEALQRVFRATLDLLVAYKNQLAEAAKETEVDYEEEMNGLQSSDDDYDDDGSDGEMDDTEEGDEAQSVKLQKLAAQAKAFHYDDDDDDDSDDDFSDEDEFQSPIDEVDAFVFFVDAIRVMQASDAQRFQNLNQSLDFTYQAIANGIAQHAELRRVEIEKEKQKKLAAASTPVTAL

SEQ ID NO: 3 - DNA, 3093 bp, Artificial Sequence
  cDNA SAD1 of Arabidopsis thaliana
atggatctgcctagcctcgctttaatcgttggcgcggctgctttcagtcccaatcctgacgagcgcagagctgctgagcagagccttaatcagttgcagcacactccacagcatttgataaggatattgcaaataattgttgatggtggctctgatctttctgtacgccaatctgctagtatccatttcaagaatttcattgctaagcactgggaacctcattctggtgaccagaacataatcttgccaagtgataagaatgttgtcaggaaccagattcttgtatttgtctctcaagtgcccccgattttgagagttcagatgggagagtgcctgaagaccataatttacgctgactatcctgagcaatggcccgaactcctggactgggtgaagcaaaacttgcaaaaaccacaagtttatggagcgctatttgtcttgaggatactatcttcaaaatacgagtttaagtcagatgaagatagggcacccattcaccgagtagtcgaggagacttttccccacctgttgaacatatttaacaatcttgtccatgttgaaaatccttctctcgaagtggcagaccatatcaagcttatttgcaagatattctggtcatgtatttatctagagcttccgagaccattgtttgatccaaacttttttaatgcttggatgggtctgttcctaaacattttggaacgtcctgttcccgtagaaggccagcctgaagaccctgagcttagaaaatcctggggatggtggaaggccaagaagtggattgctcatatcttaaacaggctatacactcggtttggagatctgaaactccagaatccagataacaaagcctttgctcaaatgttccagataaactatgcagccaagatattagagtgccacctgaaattgttgaatgccattcgtataggtggatatcttccggatagagtcatcaacctaattcttcaatatctaagcaacagtatctcgaagagtagcatgtacaacttactgcagccccaccttaacactctcctatttgaaatagttttcccacttatgtgtttcaatgataatgatcaaatgctttgggatgaagacccgcatgagtatgtaaggaagggttatgatataattgaagatttatacagtcccaggactgcgtctatggactttgtgactgagctggtcagaaagcgtggaaaagaaaacttcccaaaatttatacagttcgttgtggacatttttaagagatataatgaagcatctctggaaaacaagccttatcgccttaaggatggtgctctgcttgctgttgggacactttgtgataaacttaggcaaactgaaccctacaaatctgagctggagaatatgttagtgcaacatgtttttcctgaatttagcagcccagctggtcatcttagagctaaggctgcgtgggttgcagggcagtacgccaatatcgatttctcagaccagagcaatttttcaaaggcattgcattgtgttatctccggaatgtgtgatctagaacttcctgttcgggtggattcagtttttgctttgcgttcatttattgaagcctgcaaagatttagatgaaatccgcccagttcttcctcaacttcttgatgaatttttcaagctaatgaaggaagttgaaaacgaagaccttgcttttacattagagactattgtgtataagtttggagaggagatttctccgtatgctttgggtttgtgccaaaacttggccagtgctttctggaggtgtatcgacactgacaacggtgatgatgaaactgatgatgctggtgcattggctgcagtgggatgtctccgtgccatcagtaccattcttgaatctatcagtagtctccctcatctttatggtcagattgaaccacagttactgccaataatgcggaaaatgctgacaactgatggtcaagatgtgtttgaagaggttctggagatcgtctcatatattactactttttcacctacaatatcattggagatgtggagtctatggcctttaatgatggaagcactcgtggattgggctattgactttttcccaaatatattggtcccgttgcacaactatatatccagggggactgggcattatcttacttgcaaggaaccagattaccagcaaaatctttggaatgtgatatctgtgcttatggcgaataaaaatattgatgacagtgacctcgagccagctccgaagctattagggatagtgcttcagacatgcaagggccaggtggatcaatgggtagaaccctacctgagaatcacacttgatcggctacgcggtgctgagaaatcttccttcaagtgccttctggttgaagtggttgcaaatgccttttactacaatacccctttggcacttggcatactgcaacgatttggtatcgcaacagaaatttttactctatggttccaaatgctgcaagaaaagaagaagagtggtgcacgttctaacttcaagagagaacatgataagaaagtttgcatcttggggttgacatcacttttcagccttccagctggtcaattgcctggagaggtcttgccacatgtattcagggctcttctcgagcttttagtggcatacaaggatcaacttgctgaagcggcaaaggcagaagaggaagaagaagatgaagatggtgatgatgacgacatggatgaattccaaacagatgatgaagatgaggacggcgatgacgagaatccagatgaaactgatggaagtacactccgcaagttagcagcacaggcaaaggacttccgctcttacagcgatgatgatgacttttcagacgatgattttagcgatgacgaggaattagaatctccaattgatgaagtggatcctttcgtactcttcatggatgcagtcacagcaatgcaagtatcagactcgccaaggttccaaagccttacccagacactagacccgcactaccatggactcgcaagcacaatagctcaacacacggagctgagaagagcagagattctaaaggagaaacttgagaagcaatcttctgcaacggttgcttcctga

SEQ ID NO: 4 - protein, 1030 aa, Arabidopsis thaliana
MDLPSLALIVGAAAFSPNPDERRAAEQSLNQLQHTPQHLIRILQIIVDGGSDLSVRQSASIHFKNFIAKHWEPHSGDQNIILPSDKNVVRNQILVFVSQVPPILRVQMGECLKTIIYADYPEQWPELLDWVKQNLQKPQVYGALFVLRILSSKYEFKSDEDRAPIHRVVEETFPHLLNIFNNLVHVENPSLEVADHIKLICKIFWSCIYLELPRPLFDPNFFNAWMGLFLNILERPVPVEGQPEDPELRKSWGWWKAKKWIAHILNRLYTRFGDLKLQNPDNKAFAQMFQINYAAKILECHLKLLNAIRIGGYLPDRVINLILQYLSNSISKSSMYNLLQPHLNTLLFEIVFPLMCFNDNDQMLWDEDPHEYVRKGYDIIEDLYSPRTASMDFVTELVRKRGKENFPKFIQFVVDIFKRYNEASLENKPYRLKDGALLAVGTLCDKLRQTEPYKSELENMLVQHVFPEFSSPAGHLRAKAAWVAGQYANIDFSDQSNFSKALHCVISGMCDLELPVRVDSVFALRSFIEACKDLDEIRPVLPQLLDEFFKLMKEVENEDLAFTLETIVYKFGEEISPYALGLCQNLASAFWRCIDTDNGDDETDDAGALAAVGCLRAISTILESISSLPHLYGQIEPQLLPIMRKMLTTDGQDVFEEVLEIVSYITTFSPTISLEMWSLWPLMMEALVDWAIDFFPNILVPLHNYISRGTGHYLTCKEPDYQQNLWNVISVLMANKNIDDSDLEPAPKLLGIVLQTCKGQVDQWVEPYLRITLDRLRGAEKSSFKCLLVEVVANAFYYNTPLALGILQRFGIATEIFTLWFQMLQEKKKSGARSNFKREHDKKVCILGLTSLFSLPAGQLPGEVLPHVFRALLELLVAYKDQLAEAAKAEEEEEDEDGDDDDMDEFQTDDEDEDGDDENPDETDGSTLRKLAAQAKDFRSYSDDDDFSDDDFSDDEELESPIDEVDPFVLFMDAVTAMQVSDSPRFQSLTQTLDPHYHGLASTIAQHTELRRAEILKEKLEKQSSATVAS

SEQ ID NO: 5 - DNA, 755 bp, Artificial Sequence
  Insertion Flanking Sequence of WiscDsLox477- 480P7
  misc_feature (5)..(5): n is a, c, g, or t
  misc_feature (13)..(13): n is a, c, g, or t
  misc_feature (40)..(40): n is a, c, g, or t
  misc_feature (58)..(58): n is a, c, g, or t
  misc_feature (64)..(64): n is a, c, g, or t
  misc_feature (106)..(106): n is a, c, g, or t
  misc_feature (129)..(129): n is a, c, g, or t
  misc_feature (134)..(134): n is a, c, g, or t
  misc_feature (136)..(136): n is a, c, g, or t
  misc_feature (154)..(154): n is a, c, g, or t
  misc_feature (157)..(157): n is a, c, g, or t
  misc_feature (159)..(161): n is a, c, g, or t
  misc_feature (165)..(165): n is a, c, g, or t
  misc_feature (175)..(175): n is a, c, g, or t
  misc_feature (184)..(184): n is a, c, g, or t
  misc_feature (187)..(187): n is a, c, g, or t
  misc_feature (189)..(189): n is a, c, g, or t
  misc_feature (194)..(194): n is a, c, g, or t
  misc_feature (208)..(208): n is a, c, g, or t
  misc_feature (212)..(212): n is a, c, g, or t
  misc_feature (222)..(222): n is a, c, g, or t
cttgngttggcgnaaaggaacacgaaactcgacatatccnaatgccgacaaccaaatnccgacnagcaaatgccacatgcgaatgccgaacacgatactcgagtancgtccgcaatgtgttatgtaagntgtcntnacctttacttgagttggnatntnnngaantccggatccntaagctganctncnactgnaacaggacgttccnaaanactcaagaanagaaccatccaggcattaaagacgtttagatcaaatagttgccttggaagctccagctgcccacagagaacagtaaaaaagacattaaacagcttaactggtttgaattgtgttttaaggatatgcatattatgttaagaaagataaaatgtgaaattccgcagaggcatatgtaactgatctctctagcaacttacatatatggatgaccaaaatattttgcatatcaacttcataagttctgctatctccagagacggatttggtatctggataagcccattaaaaatagtcaaaagttgaggaaatgtctcctccacaatgcgggaaaccggtgtcctctcctcatctgacttgaacctagaaaataaaatcatgaaacatcatcaacgggatatactacttaggtgtcatcatataaagagcaacttgcatttctagctggaagtcgtcacatacttgacaggacatcatgaattgacagagacacgaaaaccattgtgttctaacaatacgttttaaacactctcatt

SEQ ID NO: 6 - protein, 1032 aa, Beta vulgaris
MDLPSLAVILRAALSPNPDERKAGEESLNQFQYTPQHTVRLLQIIVDGNCDMAVRQVASIHFKNFIAKNWSPHDPEEQSKIAPADKDMVRENILVFVAQVPTLLRVQLGECLKTIIHTDYPEQWPGLLHWVKHNLQDQQVYGALFVLRILARKYEFKSDDERTPVHLIVEETFPPLLNIFNKLVQIPNPSIDVAELIKLICKIFWSSIYLEIPKQLFEPNLFNAWMVLFLNILERPVPLEGQPADLEQRKSWGWWKVKKWTVHILNRLYTRFGDMRLQNPENKAFALMFQKSYAGKILDCHLNLLNVIRAGGYLPDRVINLILQYLSNSISKSSMYQLLQPRLDVLLFEIIFPLMCFNDNDQRLWDEDPHEYVRKGYDIIEDLYSPRTAAMDFVSELVRKRGKENLQKFIQFVVEILKRYDEIPVEHKPYRQKDGALLAIGALCDKLKQTAPYKSELERMLVQHVFSEFNSPAGHLRAKAAWVAGQYAHINFSDQDNFRKALHSVVAGLRDPELPVRVDSVFALRSFVEACKDLGEIRPILPQLLDEFFKLMNEVENEDLVFTLETIVDKFGEEMAPYAFGLCQNLAAAFWKCMSTAEADEEGDDPGGALAAVGCLRAISTILESVSRLPHLFAQIEPTLLPIMRRMLTTDGQEVFEEVLEIVSYMTFFSPTISLDMWSLWPLMMDALSDWAIDFFPNILVPLDNYVSRSTAHFLTCKDPDYQQSLWKMISSIMSDNNLEDGDIEPAPKLIEVVFQNCKGQVDHWVEPYLRITVERLRRAEKPYLKCLLIQVIANALYYNPSLALSILHQLGVATEIFNLWFQMIQQVKRSGARANFKREHDKKVCCLGLTSLLSLPADQLPVEALGRIFRVTLELLVAYKEQVAETAKEDEAEDDDEMDGFESDGEDEGDGSDKEMGVDEDEGDDADSNHLNRLAARAKAFRSTDEDDDDSEDDYSDDEELQSPIDEVDPFILFVDTVKVMQGSDPLRFQNLTQTLEFQYQALANGVAQHADQRRVEIEKEKLEKAAAAPS

SEQ ID NO: 7 - protein, 1039 aa, Brassica napus
MDLPSLALILRAAALSPNPDERKASEQQLNQLQHTPQHLVRLLQIVVDGNCDMAVRQIASIQFKNFIAKNWSPVDSGEQPRILQSDKELVRDNILVYVTHVPTLLRSQLGECLKTIIYSDYPEQWPRLLDWVKYNLQNQQIYGALFVLRILSRKYEFKSDEERTPVSRIVEETFPVLLTIFNGLIQIENPSLEIAEFMKLICKIFWSSIYLELPKQLADPNVFNAWMLLFLTVSERPVPVEGQPMDPELRKSWGWWKVKKWTVHILNRLYSRFGDPKLQTLENKPFAQMFQKTYAGRILEGHLNFLNTIRLGGYIPDRVTNLLLQYLSNSISKNSMYSLLLPRLDVLLFEIVFPLMCFNDSDQKLWEEDPHEYVRKGYNIIEDLYSPRTASMDFVNELVQKRGIENLPKFVQFVVGIFRSYDEAPAEHKPYRQKDGAMLAVGALCDKLKKTEPYKSELEHMLVQHIFPEFSSPAGHLRAKAAWVAGQYAHINFSDQNNFRKALHSVVSGMRDPDLPVRIDSVFALRSFVEACKDLNEIRPILPQLLDEFFKLMNEVENEDLVFTLETIVDKFGEEMAPFAFGLCQNLAAAFWRCLNTSEAGDDSDDMGALAAVGCLRAISTILESVSSLPQLFVEIEPTILPIMQKMLTTDGHDVFEEVLEIASYMTFYSPTISLDIWSLWPLLVEALVDWAIDFFPNILVPMDNFISRGTAHFLTCKEPDYQQSLFNVLSTLMTDRNIEDSDVESAPKLIEVVFQNCKGQVDQWVEPYLRLTVDRLQRAETSYLKSLLIQVVANMLYYNPGLTLGVLHNTGLGSTVFDLWFQMLQQKKKSGLPANFKREHDKKVCCLGLTSLLALPGGQFPDEALQRVFRATLDLLVAYKNQIAEAKEAEVDYENEMNGLDSDDEDDDGSDGEMGMDDTEDGDEAESMRLKKLAAQAKSYGYDDEDEDDDDSDDGSDDDDEFQSPIDEVDAFVFFVDAIRVMQASDAQRFQNLNQSLDFTYQAIANGIAQHAEVRRVEIEKEKQKKAAEAAGTPVPAI

SEQ ID NO: 8 - protein, 1037 aa, Brassica napus
MDLPSLALILRAAALSPNPDERKASEQQLNQLQHTPQHLVRLLQIVVDGNCDMAVRQIASIQFKNFIAKNWAPVDSGEQPRILQSDKELVRDNILVYVTQVPTLLRSQLGECLKTIIYSDYPEQWPRLLDWVKYNFQNQQIYGALFVLRILSRKYEFKSDEERTPVSRIVEETFPVLLTIFNGLIQIENPSLEIAEFMKLICKIFWSSIYLELPKQLTDPNVFNAWMLLFLTVSERPVPVEGQPMDPELRKSWGWWKVKKWTVHILNRLYSRFGDPKLQTLENKPFAQMFQKTYAGRILEGHLNFLTTIRLGGYIPDRVTNLLLQYLSNSISKNSMYSLLLPRLDVLLFEIVFPLMCFNDSDQKLWEEDPHEYVRKGYNIIEDLYSPRTASIDFVNELVQKRGKENLPKFVQFVVGIFRSYDEAPAEHKPYRQKDGAMLAVGALCDKLKKTEPYKSELEHMLVQHIFPEFSSPAGHLRAKAAWVAGQYARINFSDQNNFRKSLHSVVSGMRDPDLPVRIDSVFALRSFVEACKDLNEIRPILPQLLDEFFKLMNEVENEDLVFTLETIVDKFGEEMAPFAFGLCQNLAAAFWRCLNTSEAGDDSDDMGALAAVGCLRAISTILESVSSLPQLFVEIEPTILPIMQKMLTTDGHDVFEEVLEIASYMTFYSPTISLDIWSLWPLLVEALVDWAIDFFPNILVPMDNFISRGTAHFLTCKEPDYQQSLFNVLSTLMTDRNIEDSDVESAPKLIEVVFQNCKGQVDQWVEPYLRLTVDRLQRAETSYLKSLLIQVVANMLYYNPALTLGVLHNTGLGSTVFDLWFQMLQQKKKSGLAANFKREHDKKVCCLGLTSLLALPGGQFPDEALQRVFRATLDLLVAYKNQIAAAKEAEVDYENEMNGLDSDDEDDDGSDGEMGMDDTEDGDEAESMRLKKLAAQAKSYGYDDEDDDDSDDGSDDDDEFQSPIDEVDAFVFFVDAIRVMQASDAQRFQNLNQSLDFTYQAIANGIAQHAEVRRVEIEKEKQKKAAEAAGAPVPAI

SEQ ID NO: 9 - protein, 1040 aa, Brassica oleracea
MDLPSLALILRAAALSPNPDERKASEQQLNQLQHTPQHLVRLLQIVVDGNCDMAVRQIASIQFKNFIAKNWAPVDSGEQPRILQSDKELVRDNILVYVTQVPTLLRSQLGECLKTIIYADYPEQWPRLLDWVKYNLQNQQIYGALFVLRILSRKYEFKSDEERTPVSRIVEETFPVLLTIFNGLIQIENPSLEIAEFMKLICKIFWSSIYLELPKQLADPNVFNAWMLLFLTVSERPVPVEGQPMDPELRKSWGWWKVKKWTVHILNRLYSRFGDPKLQTLENKPFAQMFQKTYASRILEGHLNFLNTIRLGGYIPDRVTNLLLQYLSNSISKNSMYSLLLPRLDVLLFEIVFPLMCFNDSDQKLWEEDPHEYVRKGYNIIEDLYSPRTASMDFVNELVQKRGKENLPKFVQFVVGIFRSYDEAPAEHKPYRQKDGAMLAVGALCDKLKKTEPYKSELEHMLVQHIFPEFSSPAGHLRAKAAWVAGQYAHINFSDQNNFRKSLHSVVSGMRDPDLPVRIDSVFALRSFVEACKDLNEIRPILPQLLDEFFKLMNEVENEDLVFTLETIVDKFGEEMAPFAFGLCQNLAAAFWRCLNTSEAGDDSDDMGALAAVGCLRAISTILESVSSLPQLFVEIEPTILPIMQKMLTNDGHDVFEEVLEIASYMTFYSPTISLDIWSLWPLLVEALVDWAIDFFPNILVPMDNFISRGTAHFLTCKEPDYQQSLFNVLSTLMTDRNIEDSDVESAPKLIEVVFQNCKGQVDQWVEPYLRLTVDRLQRAETSYLKSLLIQVVANMLYYNPALTLGVLHNTGLGSTVFDLWFQMLQQKKKSGLAANFKREHDKKVCCLGLTSLLALPGGQFPDEALQRVFRATLDLLVAYKNQIAEAAKEAEVDYENEMNGLDSDDEDDDGSDGEMGMDDTEDGDEAESMRLKKLVAQAKSYGYDDEDEDDDDSDDGSDDDDEFQSPIDEVDAFVFFVDAIRVMQASDAQRFQNLNQSLDFTYQAIANGIAQHAEARRVEIEKEKQKKAAEAAGTPVPAI

SEQ ID NO: 10 - protein, 1039 aa, Capsicum annuum
MDLQNLAIILRGALSPNPDERKAAENSLNQFQHTPQHLVRLLQIIVDGSCDMAVRQVASIHFKNFVAKNWSPHDPAEQSKIMPSDKELVRQNILIFVAQVPSLLRVQLGECIKTMIHADYPEQWPTLLPWVKHNLQDQQVYGALFVLRILSRKYEFKSDEERAPVYHVVEETFPHLLNIFNRLVQIANPSIEVADLIKLICKIFWSSIYLEIPKQLFDPNVFNAWMVLFLNMLERPVPVEGQPADPELRKSWGWWKVKKWTIHILNRLYTRFGDLKLQNPDNKAFAQMFQKGYAGKILECHLNLLNVIRAGGYLPDRVINLILQYLSNSISKSNMYSLLQPRLDTVLFEIIFPLMCFSDNDQKLWEEDPHEYVRKGYDIIEDLYSPRTAAMDFVSELVRKRGKENLQKFILFIVEIFKRYEEAAPEYKPYRQKDGALLAIGALCDKLKQTEPYKSELERMLVQHVFPEFSSPVGHLRAKAAWVAGQYAHINFSDPNNFRKALHSVVNGMRDPDLPVRVDSVFALRSFVEACKDLNEVRPILPQLLDEFFKLMNEVENEDLVFTLETIVDKFGEEMAPYAIGLCQNLAAAFWKCINSSEAEEEADDAGALAAVGCLRAISTILESVNRLPHLFMHIEPTLLPIMRRMLTTDGQEVFEEVLEIVSYMTFFSPTISVDMWSLWPLMMEALADWAIDFFPNILVPLDNYISRSTAHFLTCKDPDYQQSLWNMISSVMGDKNLEDSDIESAPKLIQVVFQHCKGQVDQWVEPYIRITIERLRRTEKPYLKCLLVQVIADALFYNAPLTLNILQKLGVATEVFNLWFQMLGETKKSGTRANFKREQDKKVCCLGLTSLLPLPVDQLPGEALERVFKATLDLLVAYKDQVAEAAKEDEAEDDDDMNGLQTDEDDDEDDGSDKEMGDDAEEGDEADSTRLQKLAAQAKAFRSHDEDEDDDSDDDFSDDEEELQSPLDEVDPFIFFVETIKALQASDPLRFQSLTQTLDFHYQALANGVAQHAEQRKVEIEKEKMEKASAAAAAAAAS

SEQ ID NO: 11 - protein, 1029 aa, Cucumis sativus
MDLPSLAVVLQAVLSPNPDERKAAEQSLNQIQHTPQHLVRMLQIIVDNNCDLAVRQVASIHFKNYIAKNWSPVDPDEHQKISESDKDAVRKNILPFLSQVPSLLRVQLGECLKTIIHADYPEQWPSLLEWVKENLLASNVYGALFVLRILARKYEFKSDDDRTPVYRIVDETFPLLLNIFSRLVQIGDPSLEVAELIKFICKIFWSSIYMEIPKHLFDTHVFNAWMMLFLNILERPVPLEGQPADPELRKSWGWWKVKKWTVHILNRLYTRFGDLKLKNPESRAFAQAFQKNYAGKVMECHLNLLNVIRSGGYLPDRVTNLILQYLSNSISKNSMYSLLQPRLDSLLFEIIFPLMCFNDNDQKLWDEDPHEYVRKGYDIIEDLYSPRTASMDFVSELVRKRGKENLQKFIQFIVGIFNRYDEATIEFKPYRQKDGALLAIGALCDKLKQTEPYKSELERMLVQHVFPEFNSPVGHLRAKAAWVAGQYAHINFADQNNFRKALHSVVAGMRDPELPVRVDSVFALRSFVEACRDLNEIRPILPQLLDEFFKLMNEVENEDLVFTLETIVDKFGEEMAPYALGLCHNLAAAFWRCMNTAEADEEADDPGALAAVGCLRAISTILESVSRIPQLFVQIEPTLLPIMRRMLTTDGQEVFEEVLEIVSFMTFFSPTISMDMWSLWPLMMEALSEWAIDFFPNILVPLDNYVSRGTAHFLTCKAPDYQQSLWNMISSIMTDKNLEDGDIEPAPKLIQVVFQNCKGQVDQWIEPYLRITIDRLQRTEKSYLKCLLMQVISDALYYNASLSLNILQKLGVAADVFNLWFQMLQQVKKSGIRVNFRREQDKKVCCLGLTSLLALPADQLPGEALGRVFRATLDLLVAYKDQVAEAAKEEEVEEDDEMDGYPSDEDDDDGDGSDKEMGFDGEDGDEVDSIKLQKLAAQAKSFRPDDDDFDSDDDYSDDEEMQSPLDDVDPFIYFVDTIKAMQGLDPMRFQSLSQSLEFQYQALAHGVAQHAEQRRVEIEKEKLERAASS

SEQ ID NO: 12 - protein, 1026 aa, Glycine max
MDLAMILEAALSPQPDERKGAEQRLDEMQQAPQHPVSLFQIIVDSSNCNMPIRQVAAIHFKNLIAKNWTKISLDHKELLRNHILLFLPQLPPLLRSQLGECLKTIIHSDYPHHFPHLLDWIILNLQDHHHVHSSLFVLRILSRKYEFKSDDERTPIYRVVEDTFPLLLNIFNSFLQIPNPSIELADLIKLISKIFWSSIYLEVPKVLFDQNVFNAWMVLFLNVLERPVPLEGQPADPELRKSWGWWKVKKWTVHILNRLYTRFGDLKLHNLENKAFAQMFQKHYAGKILDCYLNLLNVIRVGGYLPDRVINLVLQYLSNSISKNSMYTALQPRLDVLLFEIVFPLMCFNDNDQKLWDEDPHEYVRKGYDIIEDLYSPKTASLDFVSELIRKRGKDNLHNFIQFTVEIFKRYDEAPAEYKPYRQKDGALLAIGALCDKLKQTEPYKSELEHMLVQHVFPEFSSPVGHLRAKAAWVAGQYAHINFSDQNNFRKALQCVVCGMRDPELPVRIDSVFALRSFVEACKDLNEIRPILPQLLDEFFKLMNEIENEDLVFTLETIVDKFGEEMAPYALGLCQNLAATFWRCMNTTEADDEADDPGALAAVGCLRAISTILESVSSLPQLFVQIEPALLPIMRRMLTTDGQEVFEEVLEIVSYMTFFSPTISLDMWSLWPLMMEALADWAIDFFSNILVPLDNYISRGTAHFLICKEPDYQQSLWNMISSVMGDKNMEDNDIEPAPKLIEVVFLNCKGQVDQWVEPYLRITVERLHRTEKSHLKCLLMQVIADALYYNAPLTLSILQKLGVTSEIFTLWFNMLQGVKKNGVRANFKREHDKKVCCLGLTSLLALPAGQLPGDALGQVFRATLDLLVAYKDQVAEAAKEEEAEDDDDDDMDDFQTDDEDEDDNGSDKEMGIDAEDGDEADSSKFTKLAEQTKTFRPNDEDDDDSDDDFSDDEELHSPIDEVDPFVFFVDTMKVMQSSDPLGFQNLTQTLEFSYQALANGVAQHAELRRGETEKEKLEKSSATIDS

SEQ ID NO: 13 - protein, 1027 aa, Helianthus annuus
MDLSNLVIVLKAALSPNPAERKAAEDSLNQYQYTPQHLVRLLQIVVDGNCDMAVRQAASIHFKNFIAKNWAPLDPEEQCKILPSDKDVVRQNILVFVAQLPPLLRAQLGECLKTIIHADYPEQWPGLLHWVTHNLQDQQVYGALFVLRILARKYEFKSDEERIPVYHVVEETFPHLLNIFSRLVQIANPSIEVADLIKLICKIFWSSIYLEIPKKLFDPNVFNAWMALFLNMLERPVPLEGQPIDPELRKSWGWWKVKKWTVHILNRLYTRFGDLKLQSPENKAFAQHFQKNYAGKILECHLNLLNAIRTGDYLPDRVTNLILQYLSNSISKTALYNLLQPRLDVVLFEIIFPLMCFNDNDQTLWEEDPHEYVRKGYDIIEDLYSPRTAAMDFVSELVRKRGKENLQKFILFIVEIFKRYEAASIEFKPYRQKDGALLAIGALCDKLKHTEPYKSELEPMLVQHVFPEFASPVGHLRAKAAWVAGQYAHINFSDSNNFRKALQSVVAGMRDPELPVRVDSVFALRSFVESCKDLSEIRPILPQLLDDFFKLMNEVENEDLVFTLETIVDKFGEEMAPYALGLCQNLAAAFWKCINTSEGDEEADDPGALAAAGCLRAISTILESVSRLPHLFAHTEPTLLPIMRRMLTTDGQDVFEEVLEIVSYLTFFSPTISMEMWSLWPLLLEALADWAIDFFPNILVPLDNYISRGTVHYLTCKEPDYQQSLWNVLSNIMSDKNLEDNDIEPAPKLIEVVFQNCKGQVDHWVEPYIRLTVERLRFTERPYLKCLLVQVIANALYYNPSLTLNILQKLGVATEVFNLWFQMLQQTKKSGARANFKREHDKKVCCLGLTSLLTLPSDQLPGEALERVFKATLELLVAYKDQVAEAAKDEPEDDDDMDDGLETDDDEDDMGDDAEDGDDADSLRLQRLAAQAKSFRPADDDDDDSDDEFSDDDELQSPIDEVDPFIFFMDIAKAMQASDQVRFQNLSQTLDFRYQALATGVAQHADQRRVEIEKEKLDKANAAAAAS

SEQ ID NO: 14 - protein, 1033 aa, Helianthus annuus
MDLPNLAIVLQAALSPNPAERKAAEDSLNQYQYTPQHLVRLLQITVDGNCDIAVRQAASIHFKNFIAKNWSPLDPDEQSKILPSDKDLVRQNILLFVAQVPPLLRAQLGECLKTIIHADYPEQWPGLLHWVTHNLQDQQQVYGALFVLRILARKYEFKSDEERIPVYLVVEQTFPHLLNIFGNLVQIGNPSIEVADLVKLICKIFWSSIYLEIPKKLFDPNVFNAWMVLFLNMLERPVPLEGQPADPELRKSWGWWKVKKWTVHILNRLYTRFGDLKLQNPENKAFAQHFQKNYAGKILECHLNLLNAVRTGDYLPDRVTNLILQYLSNSISKTTMYNQLQPRLDVVLFEIIFPLMCFNDNDQTLWEEDPHEYVRKGYDIIEDLYSPRTAAMDFVSELVRKRGKENLQKFILFIVEIFKRYEAAPVELKPYRQKDGALLAIGALCDKLKQTEPYKSELEPMLVQHVFPEFVSPVGHLRAKAAWVAGQYAHVNFSDPNNFRKALQCVVAGMRDPELPVRVDSVFALRSFVESCKDLGEIRPILPQLLDDFFKLMDEVENEDLVFTLETIVDKFGEEMAPYALGLCQSLAAAFWKCINTCEGDEEADDPGALAAAGCLRAISTILESVSRLPHIFAHIEPTLLPIMRRMLTTDGQDVFEEVLEIVSYMTFFSPTISMDMWSLWPLLIEALADWAIDFFPNILVPLDNYISRSTVHYLTCKEPDYQQSLWNMLSNIMSDKNLEDNDIEPAPKLIEVVFQNCKGQVDHWVEPYIRLTVERLRVAERPYLKCLLVQVIADALYYNASLTLNVLQKLGVATEVFNLWFQMLQQTKRSGGRANFKREHDKKVCCLGLTSLLTLSADQLPGEALERVFKATLELLVAYKDQVAEAAKEGSEDDDGMNDDLETDDDEDDGSDKDMGVDAEDGDEADSLRLQRLAAQAKSFRPADDDDDDDDDDFSDDEDLQSPIDEVDPFVFFMDTAKALQASDQIRFQNLSQTLDFHYQALASGISQHADQRRVEIEKEKLEKASASAPAS

SEQ ID NO: 15 - protein, 1026 aa, Helianthus annuus
MDLQSLAIVLQAALTPNPAERKAAEDTLTKFQYTPQHLVRLLQIIVDGNCDLAVRQVASIHFKNFIAENWSPHDPDEQSKILPSDKDLVRQNILVFVAQVPALLRAQLGECLKTIIHADYPEQWPALLQWVTHNLQDQQVYAALFVLRILSRKYEFKSDEERTPIHHVVEETFPHLLNIFSRLVQIGNPSIEIAGLIKLICKIYWSSIYLEIPKKLYDPNVFDAWMVLFLNILERPVPLEGQPTDPELRKSWGWWKVKKWTAHILNRLYTRFGDLKLQNPESKAFAQHFQKNYAAKILECHLNLLNAVRVGGYLPERVANLSLQYLSNSLAKSTTYSLLQTRLDVVLFEIIFPLMCFNDDDQKLWEEDPHEYVRKGYDIIEDLYSSRTAAMDFVTELVRKRGKENLQKFILFIVEIFKRYEQAPIEIKPYRQKDGALLAIGTLCDKLKQTEPYKSELEPMLVQHVFPEFTSPMGHIRAKAAWVAGQYAHINFSDPNNFRKALQSVVAGMRDPELPVRVDSVFALRSFVEACTDLGEIRPILPQLLDEFFKLMNEVENEDLVFTLETIVDKFGEEMAPYAVGLCQNLAATFWKCMNTEADDEADDPGALAAVGCLRAISTILESVSKLPHLFAHVEPTLLPIMRRMLTTDGQEVFEEVLEIASYMTFFSPTISMDMWSLWPLLMEALAEWAIDFFSNILVPLDNYISRSTVHYLTCKEPDYQQSLWNMLSNIMNDKNLEDNDIDPAPKLIAVVLQNCRGQVDQWVEPYIRITVERLHRTERPFLKCLLMQVIADALYYNPSLTLNILQKLGVATEIFNLWFQMLQQTKKNGVRVNFKRENDKKICCLGLTSLLSLSADQLPLEALERVFKATLDLLVAYKDQLTEAEKEAPEDDDDDDDDDMNDSLQTDDDDYDDDDEDGDEANSSRLQKLGAQERVFRSTDDYDDDSDDDFSDDEDLQSPIDDVDPFILFVDTVKVMQASDPTRFQNLSQTLDFRYQALANGVAQHADQRRAVIEKEKLEKASVAS

SEQ ID NO: 16 - protein, 1036 aa, Solanum lycopersicum
MDFQNLAVILAGALSPNPDERKAAENSLNQFQHTPQHLVRLLQIIVDGSCDMAVRQVASIHFKNFVAKNWCPHDPAEQSKIMPSDKELVRQNILIFIAQVPSLLRVQLGECIKTMIHADYPEQWPTLLPWIKHNLQDQQVYGALFVLRILSRKYEFKSDEERTPVYHVVEETFPHLLNIFNKLVQITNPSIEVADLIKLICKIFWSSIYLEIPKQLFDPNVFNAWMVLFLNMLERPVPVEGQPADPELRKSWGWWKVKKWTVHILNRLYTRFGDLKLQNPDNKAFAQMFQKGYAGKILECHLNLLNVIRAGGYLPDRVINLILQYLSNSISKSNMYSLLQPRLDIVLFEIIFPLMCFSDNDQKLWEEDPHEYVRKGYDIIEDLYSPRTAAMDFVSELVRKRGKENLHKFLFFIVEIFKRYQEAAPEYKPYRQKDGALLAIGALCDKLKQTEPYKSELEHMLVQHVFPEFSSPVGHLRAKAAWVAGQYAHINFADPNNFRNALHSVVTGMRDPDLPVRVDSVFALRSFIEACKDLNEIRPILPQLLDEFFKLMNEVENEDLVFTLETIVDKFGEEMAPYALGLCQNLAAAFWKCMNSAEAEEEADDPGALAAVGCLRAISTILESVSRLPHLFIHIEPTLLPIMRRMLTTDGQEVFEEVLEIVSYMTFFSPTISMDMWTLWPLMMEALADWAIDFFPNILVPLDNYISKSTAHFLTCKDPDYQQSLWNMISSVMGDKNLEDGDIESAPKLIQVVFEHCKGQVDHWVEPYIRLTVERLRRAEKSHLKCLLVQVIADALYYNAPLTWNILQKLGLALEVFNLWFLMLGQTKKSGKRVNFKREHDKKVCCLGLTSLLPLPVDQFPREALDRVFKATLDLLIAYKDQVAEAAKEDEAEDDDDMNGLQTDEDDDEDDESDKEMGDDAEEGDEADSTRLQKLAAQAKAFRSHDEDDDDDDSDDDFSDDDEEMQSPLDEVDPFIFFVETIKAMQASDPVKFQSLTQTLDFRYQALANGVAVHAEERRAEIEKEKLEKASAVVAS

SEQ ID NO: 17 - protein, 1037 aa, Sorghum bicolor
MDLPSLAVVLRAALSHVPEERKAAEESLNQFQYTPQHLVRLLQIIVDGSCDMAVRQVASIHFKNFIAKNWSPNDPDESPKVLESDKAMVRENILGFIVQVPPLLRAQLGESIKTIIHSDYPEQWPSLLHWVSHNLDLQNQIFGALYVLRVLARKYEFKSEDERIPLYRIVEETFPRLLSIFSKLVQIVNPPIEVADLIKLICKIFWSSIYLEIPKQLFDPNVFNAWMVLFINLLERPVPVEGQPIDPEIRKSWAWWKVKKWTIHILNRLYTRFGDLKLQKPESKAFAQMFQKTYAGKILACHMQLLNAIRSGDYLPDRVVNLVLQYLTNSVTKNSMYQMMQPQIDILLFEIIFPLMCFNDNDQKLWEEDPHEYVRKGYDIIEDLYSPRTAAMDFVSELVRKRGKSNLQKFIHFIVDIFRRYDEASADLKPYRQKDGALLAIGTLCDKLKQTDPYKSELERMLVQHVFPEFTSRVGHLRAKAAWVAGQYAHINFSDPNNFRQAMHCIVSGMRDPDLPVRVDSVFALRSFVEACKDLNEIRPILPQLLDEFFKLMNEVENEDLVFTLETIVDKFGEEMAPYALGLCQNLAAAFWRCMASSETDDEADDSGALAAVGCLRAISTILESISSLPHLFMQIEPTLLPIMRRMLTSDGQDVYEEVLEIVSYMTFFSPTISLDMWSLWPLMMEALNDWAIDFFENILVPLDNYISRGTDHFLECKDPDYQQSLWNALQSIMMDENMEDSDIEPAPKLIEVVFQNCKGNVDQWVEHYLRITIERLRRAKKPYLKCLLVQVIANALYYNPALTLETLNKLGVAADIFNHWFAMLQQVKKSGARVNFKREHDKKVCCLGLTSLIALPADKIPAEALDRIFKATLGLFFPSKLCLCFFSPGEYTEAKKQNEEGADDMDGFDADEEDDDEVDSDKEMGLDDEDGDEVSSLQLQKLAAEARGFQPADEDDDSDDDFSDDEELHSPIDEVDPFIFFVETVQGLQASDPARFQNLMQTLDFRYQALASGIAQHAEERKIEIEKEKSEKANAQ

SEQ ID NO: 18 - protein, 1027 aa, Zea mays
MDLPSLAVVLRAALSHVPEERKAAEESLNQFQYTPQHLVRLLQIIVDGSCDMAVRQVASIHFKNFIAKNWSPNDPDESPKVLESDKAMVRENILGFIVQVPPLLRAQLGESIKTIVHSDYPEQWPSLLHWVSHNLDLQNQIFGALYVLRVLARKYEFKSEDERIPLFHIVEETFPRLLSIFSKLVQIVNPPIEVADLIKLICKIFWSSIYLEIPKQLFDPNVFNAWMVLFINLLERPVPVEGQPIDPEIRKSWAWWKVKKWTIHILNRLYTRFGDLKLQKPESKAFAQMFQKTYSGKILACHMQLLNAIRGGDYLPDRVINLVLQYLTNSVTKNSMYQMMQPQIDILLFEIIFPLMCFNDNDQKLWDEDPHEYVRKGYDIIEDLYSPRTAAMDFVSELVRKRGKNNLQKFIHFIVDIFRRYDEASADLKPYRQKDGALLSIGTLCDKLKQTDPYKSELETMLVRHVFPEFSSRVGHLRAKAAWVAGQYAHINFSDPNNFRQAMHCIVSGMHDPDLPVRVDSVFALRSFVEACKDLNEIRPILPQLLDEFFKLMNEVENEDLVFTLETIVDKFGEEMAPYALGLCQNLAAAFWRCMASSEADDEADDSGALAAVGCLRAISTILESISSLPHLFTQIEPTLLPIMRRMLTSDGQDVYEEVLEIVSYMSFYSPTISLDMWTLWPVMMEALNDWAIDFFENILVPLDNYISRGTDHFLACKDPDYQQSLWKALQSIMMDENMEDSDIEPAPKLIEVVFQNCKGNVDHWVEHYLRITIERLRRAHKPYLKCLLVQVIANALYYNPALTLEILNKLGVAADIFNHWFAMLRQVKKSGARVNFKREHDKKVCCLGLTSLIALPAAKIPADALDRIFKATLELLVAYKDQVAEAKKQNEEAADDMDGFDADEEDDDEVDSDKEMGLDDEDGDEVSSLQLQKLAAEARGFQPADDDDSDDDFSDDELHSPIDEVDPFIFFVETVQGLQASDPARFQNLMHTLDFSYQALASGIAQHAEQRKNEIEKEKSEKANAQ

SEQ ID NO: 19 - protein, 39 aa, Artificial Sequence
  consensus sequence
  misc_feature (5)..(5): Xaa can be any naturally occurring amino acid
  misc_feature (12)..(12): Xaa can be any naturally occurring amino acid
  misc_feature (14)..(14): Xaa can be any naturally occurring amino acid
  misc_feature (17)..(17): Xaa can be any naturally occurring amino acid
  misc_feature (20)..(20): Xaa can be any naturally occurring amino acid
  misc_feature (32)..(32): Xaa can be any naturally occurring amino acid
LFEIXFPLMCFXDXDQXLWXEDPHEYVRKGYXIIEDLYS

SEQ ID NO: 20 - protein, 1035 aa, Nicotiana tabacum
MDLQNLAIVLRGALSPNPDERKAAEESLNQFQYTPQHLVRLLQIIVDGSCDMAVRQVASIHFKNFVAKNWSPHDPAEQSKILLSDKELVRQNILIFIAQVPSLLRVQLGECLKTMIHADYPEQWPTLLPWVKHNLQDQQVYGALFVLRILSRKYEFKSDEERTPVYHIVEETFPQLLNIFNRLAQIANPSIEVADLIKLICKIFWSSIYLEIPKQLFDPNMFNAWMVLFLNMLERPVPLEGQPADPELRKSWGWWKVKKWTVHILNRLYTRFGDLKLQNPDNKAFAQMFQKGYAGKILECHLNLLNVIRAGGYLPDRVINLILQYLSNSISKSNMYSLLQPRLDIVLFEIIFPLMCFSDNDQKLWEEDPHEYVRKGYDIIEDLYSPRTAAMDFVSELVRKRGKENLQKFILFIVEIFKRYEEAAPEYKPYRQKDGALLAIGALCDKLKQTEPYKSELERMLVQHVFPEFSSPVGHLRAKAAWVAGQYAHVNFSDPNNFRKALHSVVAGMRDPDLPVRVDSVFALRSFVEACRDLDEIRPIIPQLLDEFFKLMNEVENEDLVFTLETIVDKFGEEMAPYALGLCQNLAAAFWKCINTSEADEEGDDPGALAAVGCLRAISTILESVSRLPHLFIHIEPTLLPIMRRMLTTDGQEVFEEVLEIVSYMTFFSPTISMEMWSLWPLMMEALADWAIDFFPNILVPLDNYISRSTAHFLTCKDPDYQKSLWNMISTVMGDKNLEDGDIEPAPKLIQVVFQHCKGQVDHWVEPYIRITIERLRRAEKPYLKCLLMQVIADALYYNASLTLNILQKLGIATEVFNLWFQMLGQKKKSGARVNFKREHDKKVCCLGLTSLLPLPVDQLPGEALERVFKASLELLVAYKDQVAEAAKEEEVEDDDDMHGLQTDEDDDEDDGSDKEMGVDAEEGDEADSTRLQKLAAQAKAFRSHDSDEDDDSDDDFSDDDEELQSPLDEVDPFIFFVETIKAMQASDPLRFQSLTQTLDFHYQALANGVAQHAEQRRVEIEKEKMEKASATAAS

SEQ ID NO: 21 - protein, 1030 aa, Oryza sativa
MDLQSLAVVLRAALSHVPEERKAAEESLNQFQYAPQHLVRLLQIIVDGSCDMAVRQVASIHFKNFVAKNWSPNDPEESQKISESDKLMVRENILGFIVQVPPLLRAQLGESIKTIIHSDYPEQWPVLLHWVTHNLESENQIFGALYVLRVLSRKYEFKSEEERIPLYHIVEETFPRLLSIFSKLVQIVNPPIEVADLIKLICKIFWSSIYLEIPKQLFDPNVFNAWMSLFINLLERPVPVEGQPLDPDVRKSWGWWKVKKWTIHILNRLYTRFGDMKLQKPESKAFAQMFQKNYAGRILGCHLQILNAVRTGDYLPDRVINLVLQYLTNSVTKNSMYQLMQPQIDIILFEIIFPLMCFNDNDQKLWDEDPHEYVRKGYDIIEDLYSPRTAAMDFVSELVRKRGKSNLQKFIHFIVDIFRRYDEASIEIKPYRQKDGALLAIGTLCDKLKQTDPYKAELERMLVQHVFPEFNSHVGHLRAKAAWVAGQYAHISFSDQDNFRKAMHCIVSGMRDPDLPVRVDSVFALRSFVEACKDLNEIRPILPQLLDEFFKLMNEVENEDLVFTLETIVDKFGEEMAPYALGLCQNLAAAFWRCMASQEADDEADDSGALAAVGCLRAISTILESVSSLPHLFIQIEPTLLPIMRRMLTSDGQDVYEEVLEIVSYMTFFSPSISLDMWSLWPLMMEALNDWAIDFFENILVPLDNYVSRGSDHFLACKNPDYQQSLWSALSSIMMDQNMEDSDIEPAPKLIEVVFQNCKGNVDQWVEPYLSLTIDRLRRAHKPYLKCLLVQVIANAFYYNPSLTLATLHKLGAVTEIFNIWFGMLEQVKKSGVRANFKREHDKKVCCLGLTSLISLPADHIPGEALNRIFKATLDLLVAYKEQVAESKKQDDADGDDMDGFDADEDEDDDEVESDKEMGLDEEDADEVNSLHLQKLAAEARGFQPADEYDDSDDDFSDDEELQSPIDEVDPFILFVETVQGLQASDPIRFQSLMQTLDFRYQALASGIAQHAEERRVEIEKEKLEKANAQ

SEQ ID NO: 22 - protein, 1030 aa, Oryza sativa
MDLQSLAVVLRAALSHVPEERKAAEESLNQFQYAPQHLVRLLQIIVDGSCDMAVRQVASIHFKNFVAKNWSPNDPEESQKISESDKLMVRENILGFIVQVPPLLRAQLGESIKTIIHSDYPEQWPGLLHWVTHNLESENQIFGALYVLRVLSRKYEFKSEDERIPLYHIVEETFPRLLSIFSKLVQIVNPPIEVADLIKLICKIFWSSIYLEIPKQLFDPNVFNAWMSLFINLLERPVPVEGQPLDPDVRKSWGWWKVKKWTIHILNRLYTRFGDMKLQKPESKAFAQMFQKNYAGRILGCHLQILNAVRTGDYLPDRVINLVLQYLTNSVTKNSMYQLMQPQIDIILFEIIFPLMCFNDNDQKLWDEDPHEYVRKGYDIIEDLYSPRTAAMDFVSELVRKRGKSNLQKFIHFIVDIFRRYDEASIEIKPYRQKDGALLAIGTLCDKLKQTDPYKAELERMLVQHVFPEFNSHVGHLRAKAAWVAGQYAHISFSDQDNFRKAMHCIVSGMRDPDLPVRVDSVFALRSFVEACKDLNEIRPILPQLLDEFFKLMNEVENEDLVFTLETIVDKFGEEMAPYALGLCQNLAAAFWRCMASQEADDEADDSGALAAVGCLRAISTILESVSSLPHLFIQIEPTLLPIMRRMLTSDGQDVYEEVLEIVSYMTFFSPSISLDMWSLWPLMMEALNDWAIDFFENILVPLDNYVSRGSDHFLACKNPDYQQSLWSALSSIMMDQNMEDSDIEPAPKLIEVVFQNCKGNVDQWVEPYLSLTIDRLRRAHKPYLKCLLVQVIANAFYYNPSLTLATLHKLGAVTEIFNIWFGMLEQVKKSGVRANFKREHDKKVCCLGLTSLISLPADHIPGEALNRIFKATLDLLVAYKEQVAESKKQDDADGDDMDGFDADEDEDDDEVESDKEMGLDEEDADEVNSLHLQKLAAEARGFQPADEYDESDDDFSDDEELQSPIDEVDPFILFVETVQGLQASDPIRFQSLMQTLDFRYQALASGIAQHAEERRVEIEKEKLEKANAQ

SEQ ID NO: 23 - protein, 1034 aa, Coffea arabica
MDLQNLAIILRGALSPNPDERKAAEESLNQFQYTPQHLVRLLQIIVDGNCDMAVRQVASIHFKNFIARNWAPHDPGEQSKILPSDKDVVRQNILNFIAQVPQLLRVQLGECLKTIIHADYPEQWPALLHWVKLNLQDQQVFGALFVLRILARKYEFKSDEERTPVYHIVDETFPPLLNIFNRLVQITNPSIEVADLIKLICKIFWSCIYLEIPKQLFDPNVFNAWMVLFLNVLERPVPLEGQPVDPEVRKSWGWWKVKKWTVHILNRLYTRFGDLKLQNPDNKVFAQMFQKNYAGKILECHLNLLNVIRVGGYLPDRVINLILQYLSNSISRSNMYNLLQPRLDVVLFEIIFPLMCFSDADQKLWDEDPHEYVRKGYDIIEDLYSPRTAAMDFVSELVRKRGKENLQKFILFIVEIFNRYNEAVPEYKPFRQKDGALLAIGALCDKLKQTEPYKSELERMLVQHVFPEFSSPAGHLRAKAAWVAGQYAHVNFSDPNNFRNALRSVVAGMRDPELPVRVDSVFALRSFVEACKDLDEIRPILPQLLDEFFKLMNEVENEDLVFTLETIVDKFGEEMAPYALGLCQNLAAAFWKCLNTAEADDEADDPGALAAVGCLRAISTILESVSSLPHLFVQIEPTLLPIMRRMLTTDGQEVFEEVLEIVSYMTFFSPTISMDMWSLWPLMMEALADWAIDFFPNILVPLDNYVSRSTVHFLTCKEPDYQQSLWHMISSIMADKNLEDGDIEPAPKLIQVVFQNCRGQVDHWVEPYIRIAVERLRRTEKPYLKCLLIEVIADALYYNASLTLSILHKLGVASDIFALWFQMLQQTKKNGVRANFKREHDKKVCCLGMTSLLSLPNDQFPAEALERVFKATLELLVAYKEQLAETEKEAAEDNDDMDGLPTDDEDDDGDVSDKEMGIDDEDGDEADSIKLQKLAAQAKAFRSTEDDEDDSDDDFSDDEELQSPIDDVDPFVFFVDTIKALQASDPLRFQNLTQTLDFHFQALANGVAQHAEQRRVEIEKEKMEKASSAAVGAS

SEQ ID NO: 24 - protein, 1031 aa, Triticum aestivum
MDLSNLTLVLRAALSHAPEERKAAEASLEQLQYTQQHLVRLLQIIVDGSCDMAVRQVASIHFKNFVSKAWSPIDPDETRKIPEGDKSMVRENILGFVTQLPPLLRAQLGESIKTLILADYPEQWPSLLHWVTHNMESQDQIFGALYVLRILSRKYEFKSEEERIPLYQIVEECFPRLLNIFSTLVQIVNPPIEVADLIKLICKIFWSSIYLEIPKQLFEPNIFNAWIVLFLNLLERPVPLEGQPSDPDARKAWGWWKVKKWITHILNRLYSRFADMKVHKPESKAFAQMFQKNYAGKILGCHLQLLNAIRTGGYLPDRVINLILQYLTNSLTKNSMYQLMQPQIDIILFEIIFPLMCFNDNDQMLWDEDPHEYVRKGYDIIEDLYSPRTAAMDFVSELVRKRGKGNLQKFIQFIVEIFMRYNEASIEVKPYRQKDGALLAIGTLCDRLKQTDPYKAELERMLVQHVFPEFSSHVGHLRAKAAWVAGQYAHITFSDQDNFRKAMHCVISGLRDPELPVRVDSVFALRSFVEACKDLDEIRPILPQLLDEFFKLMGEVENEDLVFTLETIVDRFGEEMAPYALGLCQSLAAAFWRCMASSEADDEVEDSGALAAVGCLRALSTILESISSLPHLFIQIEPTLLPILRRMLTSDGQDVYEEVLEIVSYLTFYSPTISLDMWSLWPLMMEALNDWAIDFFENILVPLDNYISRGTEHFVTCKDPDYQQCLWKGLSSVMTDQNMEDSDIVPAPKLIEVFFQNCKGQVDHWVEPYLRLTIDRLRRAEKPYLKSLLVQVIANVFYYNPSLTLAMLHKLGVATEIFNLWFVMLQQVKKSGKRVNFKREHDKKVCCLGLTSLIGLPANHIPAEALERIFKATLELLVAYKEQVAESKRQNAAAADDVDEFDADEEENEEDEDDGEMGVDDEDQDEVNSLNIQKLVQARGFQLHDEDDDDDDSDDDFSDDEELQTPIDEVDPFIFFVGTIQAVQASDPARFQSLMQTLDFHYQALANGVAQHAEERKVEIEKEKLEKANAQ

SEQ ID NO: 25 - protein, 86 aa, Artificial Sequence
  consensus sequence
  misc_feature (2)..(3): Xaa can be any naturally occurring amino acid
  misc_feature (5)..(6): Xaa can be any naturally occurring amino acid
  misc_feature (11)..(11): Xaa can be any naturally occurring amino acid
  misc_feature (19)..(19): Xaa can be any naturally occurring amino acid
  misc_feature (21)..(21): Xaa can be any naturally occurring amino acid
  misc_feature (23)..(23): Xaa can be any naturally occurring amino acid
  misc_feature (28)..(28): Xaa can be any naturally occurring amino acid
  misc_feature (46)..(46): Xaa can be any naturally occurring amino acid
  misc_feature (71)..(71): Xaa can be any naturally occurring amino acid
  misc_feature (73)..(73): Xaa can be any naturally occurring amino acid
  misc_feature (78)..(78): Xaa can be any naturally occurring amino acid
  misc_feature (81)..(81): Xaa can be any naturally occurring amino acid
GXXDXXLPVRXDSVFALRXFXEXCKDLXEIRPILPQLLDEFFKLMXEVENEDLVFTLETIVDKFGEEMAPXAXGLCQXLAXFWRCM

SEQ ID NO: 26 - protein, 44 aa, Artificial Sequence
  consensus sequence
  misc_feature (6)..(6): Xaa can be any naturally occurring amino acid
  misc_feature (11)..(11): Xaa can be any naturally occurring amino acid
  misc_feature (13)..(15): Xaa can be any naturally occurring amino acid
  misc_feature (20)..(20): Xaa can be any naturally occurring amino acid
  misc_feature (28)..(28): Xaa can be any naturally occurring amino acid
  misc_feature (37)..(37): Xaa can be any naturally occurring amino acid
  misc_feature (40)..(40): Xaa can be any naturally occurring amino acid
  misc_feature (42)..(42): Xaa can be any naturally occurring amino acid
ERPVPXEGQPXDXXXRKSWXWWKVKKWXVHILNRLYXRFXDXKL